SEQUENCE LISTING (16 sequences)

SEQ ID NO: 1 - protein, 312 aa, Artificial Sequence
  E.coli BL21(DE3) FruK WT amino acid sequence
MSRRVATITLNPAYDLVGFCPEIERGEVNLVKTTGLHAAGKGINVAKVLKDLGIDVTVGGFLGKDNQDGFQQLFSELGIANRFQVVQGRTRINVKLTEKDGEVTDFNFSGFEVTPADWERFVTDSLSWLGQFDMVCVSGSLPSGVSPEAFTDWMTRLRSQCPCIIFDSSREALVAGLKAAPWLVKPNRRELEIWAGRKLPEMKDVIEAAHALREQGIAHVVISLGAEGALWVNASGEWIAKPPSVDVVSTVGAGDSMVGGLIYGLLMRESSEHTLRLATAVAALAVSQSNVGITDRPQLAAMMARVDLQPFN

SEQ ID NO: 2 - DNA, 939 bp, Artificial Sequence
  mutant FruK A39S nucleotide sequence
atgagcagacgtgttgctactatcacccttaatccggcttatgaccttgttggtttctgcccggaaattgaacgcggcgaagtgaacctggtgaaaaccaccggtctgcatgcgtcgggtaaaggcatcaacgtggccaaagtattaaaagacctgggaattgatgtcaccgttggcggcttcctcggtaaagacaatcaggatggttttcagcaactgttcagcgagctgggcattgccaaccgtttccaggttgtacaggggcgcactcgaattaacgttaagctgacggaaaaagacggcgaagtgaccgacttcaacttctcgggttttgaagtcacccccgccgactgggaacgctttgtgactgattctctgagctggctcggtcagttcgatatggtctgtgtcagcggaagcttaccgtcaggcgtcagcccggaagcgttcaccgactggatgactcgcctgcgtagtcagtgtccttgcattatctttgatagtagccgtgaagcgttagtagcaggtttgaaagcggcaccgtggctggtgaaacctaaccgccgcgagctggaaatctgggcaggccgtaaactgcctgaaatgaaagatgtgattgaagctgcgcatgcgctgcgtgaacaaggtatcgcgcatgttgttatttcactgggtgccgaaggcgcgctttgggttaatgcctccggcgaatggatcgccaaaccaccgtcagtcgatgtcgtaagcaccgttggcgcaggggattctatggttggtggcctgatttatggcttgctgatgcgtgaatccagtgaacacacactgcgtctggcgacagctgttgcagccctggcggtaagtcaaagcaatgtgggtattaccgatcgtccgcagttggccgcaatgatggcgcgcgtcgacttacaaccttttaactga

SEQ ID NO: 3 - DNA, 67 bp, Artificial Sequence
  Cra binding site deleted nucleotide sequence
tgaaacgattcagcctctatgagaaaaaaagcgccaacctggcttagggttaaagacaagatcgcgc

SEQ ID NO: 4 - DNA, 279 bp, Artificial Sequence
  Mutated T7RNAP core promoter region
gcaaaccgcctctccccgcgcgttggccgattcattaatgcagctggcacgacaggtttcccgactggaaagcgggcagtgagcgcaacgcaattaatgtaagttagctcactcattaggcaccccaggctttacactttatgcttccggctcgtatgttgtgtgaaattgtgagcggataacaatttcacacaggaaacagctatgaccatgattacggattcactggccgtcgttttacaacgtcgtgactgggaaaaccctggcgttacccaactt

SEQ ID NO: 5 - protein, 359 aa, Artificial Sequence
  fbaA WT amino acid
MSKIFDFVKPGVITGDDVQKVFQVAKENNFALPAVNCVGTDSINAVLETAAKVKAPVIVQFSNGGASFIAGKGVKSDVPQGAAILGAISGAHHVHQMAEHYGVPVILHTDHCAKKLLPWIDGLLDAGEKHFAATGKPLFSSHMIDLSEESLQENIEICSKYLERMSKIGMTLEIELGCTGGEEDGVDNSHMDASALYTQPEDVDYAYTELSKISPRFTIAASFGNVHGVYKPGNVVLTPTILRDSQEYVSKKHNLPHNSLNFVFHGGSGSTAQEIKDSVSYGVVKMNIDTDTQWATWEGVLNYYKANEAYLQGQLGNPKGEDQPNKKYYDPRVWLRAGQTSMIARLEKAFQELNAIDVL

SEQ ID NO: 6 - protein, 477 aa, Artificial Sequence
  ptsG WT amino acid
MFKNAFANLQKVGKSLMLPVSVLPIAGILLGVGSANFSWLPAVVSHVMAEAGGSVFANMPLIFAIGVALGFTNNDGVSALAAVVAYGIMVKTMAVVAPLVLHLPAEEIASKHLADTGVLGGIISGAIAAYMFNRFYRIKLPEYLGFFAGKRFVPIISGLAAIFTGVVLSFIWPPIGSAIQTFSQWAAYQNPVVAFGIYGFIERCLVPFGLHHIWNVPFQMQIGEYTNAAGQVFHGDIPRYMAGDPTAGKLSGGFLFKMYGLPAAAIAIWHSAKPENRAKVGGIMISAALTSFLTGITEPIEFSFMFVAPILYIIHAILAGLAFPICILLGMRDGTSFSHGLIDFIVLSGNSSKLWLFPIVGIGYAIVYYTIFRVLIKALDLKTPGREDATEDAKATGTSEMAPALVAAFGGKENITNLDACITRLRVSVADVSKVDQAGLKKLGAAGVVVAGSGVQAIFGTKSDNLKTEMDEYIRNH

SEQ ID NO: 7 - protein, 269 aa, Artificial Sequence
  Escherichia coli BL21(DE3) agaR WT amino acid
MSNTDASGEKRVTGTSERREQIIQRLRQQGSVQVNDLSALYGVSTVTIRNDLAFLEKQGIAVRAYGGALICDSTTPSVEPSVEDKSALNTAMKRSVAKAAVELIQPGHRVILDSGTTTFEIARLMRKHTDVIAMTNGMNVANALLEAEGVELLMTGGHLRRQSQSFYGDQAEQSLQNYHFDMLFLGVDAIDLERGVSTHNEDEARLNRRMCEVAERIIVVTDSSKFNRSSLHKIIDTQRIDMIIVDEGIPADSLEGLRKAGVEVILVGE

SEQ ID NO: 8 - DNA, 1080 bp, Artificial Sequence
  fbaA WT gene
atgtctaagatttttgatttcgtaaaacctggcgtaatcactggtgatgacgtacagaaagttttccaggtagcaaaagaaaacaacttcgcactgccagcagtaaactgcgtcggtactgactccatcaacgccgtactggaaaccgctgctaaagttaaagcgccggttatcgttcagttctccaacggtggtgcttcctttatcgctggtaaaggcgtgaaatctgacgttccgcagggtgctgctatcctgggcgcgatctctggtgcgcatcacgttcaccagatggctgaacattatggtgttccggttatcctgcacactgaccactgcgcgaagaaactgctgccgtggatcgacggtctgttggacgcgggtgaaaaacacttcgcagctaccggtaagccgctgttctcttctcacatgatcgacctgtctgaagaatctctgcaagagaacatcgaaatctgctctaaatacctggagcgcatgtccaaaatcggcatgactctggaaatcgaactgggttgcaccggtggtgaagaagacggcgtggacaacagccacatggacgcttctgcactgtacacccagccggaagacgttgattacgcatacaccgaactgagcaaaatcagcccgcgtttcaccatcgcagcgtccttcggtaacgtacacggtgtttacaagccgggtaacgtggttctgactccgaccatcctgcgtgattctcaggaatatgtttccaagaaacacaacctgccgcacaacagcctgaacttcgtattccacggtggttccggttctactgctcaggaaatcaaagactccgtaagctacggcgtagtaaaaatgaacatcgataccgatacccaatgggcaacctgggaaggcgttctgaactactacaaagcgaacgaagcttatctgcagggtcagctgggtaacccgaaaggcgaagatcagccgaacaagaaatactacgatccgcgcgtatggctgcgtgccggtcagacttcgatgatcgctcgtctggagaaagcattccaggaactgaacgcgatcgacgttctgtaa

SEQ ID NO: 9 - DNA, 1434 bp, Artificial Sequence
  ptsGV12F gene
atgtttaagaatgcatttgctaacctgcaaaagttyggtaaatcgctgatgctgccggtatccgtactgcctatcgcaggtattctgctgggcgtcggttccgcgaatttcagctggctgcccgccgttgtatcgcatgttatggcagaagcaggcggttccgtctttgcaaacatgccactgatttttgcgatcggtgtcgccctcggctttaccaataacgatggcgtatccgcgctggccgcagttgttgcctatggcatcatggttaaaaccatggccgtggttgcgccactggtactgcatttacctgctgaagaaatcgcctctaaacacctggcggatactggcgtactcggagggattatctccggtgcgatcgcagcgtacatgtttaaccgtttctaccgtattaagctgcctgagtatcttggcttctttgccggtaaacgctttgtgccgatcatttctggcctggctgccatctttactggcgttgtgctgtccttcatttggccgccgattggttctgcaatccagaccttctctcagtgggctgcttaccagaacccggtagttgcgtttggcatttacggtttcatcgaacgttgcctggtaccgtttggtctgcaccacatctggaacgtacctttccagatgcagattggtgaatacaccaacgcagcaggtcaggttttccacggcgacattccgcgttatatggcgggtgacccgactgcgggtaaactgtctggtggcttcctgttcaaaatgtacggtctgccagctgccgcaattgctatctggcactctgctaaaccagaaaaccgcgcgaaagtgggcggtattatgatctccgcggcgctgacctcgttcctgaccggtatcaccgagccgatcgagttctccttcatgttcgttgcgccgatcctgtacatcatccacgcgattctggcaggcctggcattcccaatctgtattcttctggggatgcgtgacggtacgtcgttctcgcacggtctgatcgacttcatcgttctgtctggtaacagcagcaaactgtggctgttcccgatcgtcggtatcggttatgcgattgtttactacaccatcttccgcgtgctgattaaagcactggatctgaaaacgccgggtcgtgaagacgcgactgaagatgcaaaagcgacaggtaccagcgaaatggcaccggctctggttgctgcatttggtggtaaagaaaacattactaacctcgacgcatgtattacccgtctgcgcgtcagcgttgctgatgtgtctaaagtggatcaggccggcctgaagaaactgggcgcagcgggcgtagtggttgctggttctggtgttcaggcgattttcggtactaaatccgataacctgaaaaccgagatggatgagtacatccgtaaccactaa

SEQ ID NO: 10 - DNA, 810 bp, Artificial Sequence
  Escherichia coli BL21(DE3) agaR WT gene
atgagtaataccgacgcttcaggtgagaagcgagtgacaggcaccagcgagcgacgagaacagatcattcagcgtctgcgacagcaagggagtgtgcaggttaacgatctgtcggcattgtatggcgtatctaccgtgacgatccgcaacgatctggcgtttctggaaaagcaggggatcgctgtgcgtgcctatggtggcgcgttgatctgcgatagcacgacgccgtcagtcgagccatcagtggaagataaaagcgcactgaacaccgcgatgaaacgcagcgttgcgaaagctgccgttgagttgattcagccaggtcatcgggtgatcctcgattccgggaccaccacttttgagattgctcgtctgatgcgcaagcacactgacgtaattgcgatgaccaacggtatgaacgtggctaatgctttgctggaagcggaaggcgttgagctgctgatgaccggcgggcatttgcgccgtcagtcgcaatctttttacggcgatcaggctgaacaatcgctgcaaaattaccacttcgatatgctgtttcttggtgtagatgcgatcgatctggagcgcggcgtcagcacgcataatgaagatgaagcccgtttaaaccgccggatgtgcgaagttgcggaacggatcatcgtagtcaccgattccagtaagttcaaccgctccagtttacataagatcattgatactcaacgtatcgacatgatcattgttgatgaaggcattcctgcggatagtctggaaggactgcgaaaggctggggttgaagtgattctggtcggggagtga

SEQ ID NO: 11 - protein, 481 aa, Artificial Sequence
  uxaE
MVLKVFKDHFGRGYEVYEKSYREKDSLSFFLTKGEEGKILVVAGEKAPEGLSFFKKQRVEGVSFFFCERNHENLEVLRKYFPDLKPVRAGLRASFGTGDRLGITTPAHVRALKDSGLFPIFAQQSVRENERTGRTWRDVLDDATWGVFQEGYSEGFGADADHVKRPEDLVSAAREGFTMFTIDPSDHVRNLSKLSEREKNEMFEEILKKERIDRIYLGKKYTVLGERLEFDEKNLRDAALVYYDAIAHVDMMYQILKDETPDFDFEVSVDETETPTSPLFHIFVVEELRRRGVEFTNLALRFIGEWEKGIDYKGDLAQFEREIKMHAEIARMFEGYKISLHSGSDKFSVYPAFASATGGLFHVKTAGTSYLEAVKVISMVNPELFREIYRCALDHFEEDRKSYHISADLSKVPEVEKVKDEDLPGLFEDINVRQLIHVTYGSVLKDASLKERLFKTLEQNEELFYETVAKHIKRHVDLLKG

SEQ ID NO: 12 - protein, 312 aa, Artificial Sequence
  Synthetic FruK A39S mutant polypeptide
MSRRVATITLNPAYDLVGFCPEIERGEVNLVKTTGLHASGKGINVAKVLKDLGIDVTVGGFLGKDNQDGFQQLFSELGIANRFQVVQGRTRINVKLTEKDGEVTDFNFSGFEVTPADWERFVTDSLSWLGQFDMVCVSGSLPSGVSPEAFTDWMTRLRSQCPCIIFDSSREALVAGLKAAPWLVKPNRRELEIWAGRKLPEMKDVIEAAHALREQGIAHVVISLGAEGALWVNASGEWIAKPPSVDVVSTVGAGDSMVGGLIYGLLMRESSEHTLRLATAVAALAVSQSNVGITDRPQLAAMMARVDLQPFN

SEQ ID NO: 13 - DNA, 939 bp, Artificial Sequence
  FruK WT sequence
atgagcagacgtgttgctactatcacccttaatccggcttatgaccttgttggtttctgcccggaaattgaacgcggcgaagtgaacctggtgaaaaccaccggtctgcatgcggcgggtaaaggcatcaacgtggccaaagtattaaaagacctgggaattgatgtcaccgttggcggcttcctcggtaaagacaatcaggatggttttcagcaactgttcagcgagctgggcattgccaaccgtttccaggttgtacaggggcgcactcgaattaacgttaagctgacggaaaaagacggcgaagtgaccgacttcaacttctcgggttttgaagtcacccccgccgactgggaacgctttgtgactgattctctgagctggctcggtcagttcgatatggtctgtgtcagcggaagcttaccgtcaggcgtcagcccggaagcgttcaccgactggatgactcgcctgcgtagtcagtgtccttgcattatctttgatagtagccgtgaagcgttagtagcaggtttgaaagcggcaccgtggctggtgaaacctaaccgccgcgagctggaaatctgggcaggccgtaaactgcctgaaatgaaagatgtgattgaagctgcgcatgcgctgcgtgaacaaggtatcgcgcatgttgttatttcactgggtgccgaaggcgcgctttgggttaatgcctccggcgaatggatcgccaaaccaccgtcagtcgatgtcgtaagcaccgttggcgcaggggattctatggttggtggcctgatttatggcttgctgatgcgtgaatccagtgaacacacactgcgtctggcgacagctgttgcagccctggcggtaagtcaaagcaatgtgggtattaccgatcgtccgcagttggccgcaatgatggcgcgcgtcgacttacaaccttttaactga

SEQ ID NO: 14 - DNA, 279 bp, Artificial Sequence
  Wt T7RNAP core promoter region
gcaaaccgcctctccccgcgcgttggccgattcattaatgcagctggcacgacaggtttcccgactggaaagcgggcagtgagcgcaacgcaattaatgtaagttagctcactcattaggcaccccaggctttacactttatgcttccggctcgtataatgtgtggaattgtgagcggataacaatttcacacaggaaacagctatgaccatgattacggattcactggccgtcgttttacaacgtcgtgactgggaaaaccctggcgttacccaactt

SEQ ID NO: 15 - protein, 477 aa, Artificial Sequence
  Synthethic ptsGV12F polypeptide
MFKNAFANLQKFGKSLMLPVSVLPIAGILLGVGSANFSWLPAVVSHVMAEAGGSVFANMPLIFAIGVALGFTNNDGVSALAAVVAYGIMVKTMAVVAPLVLHLPAEEIASKHLADTGVLGGIISGAIAAYMFNRFYRIKLPEYLGFFAGKRFVPIISGLAAIFTGVVLSFIWPPIGSAIQTFSQWAAYQNPVVAFGIYGFIERCLVPFGLHHIWNVPFQMQIGEYTNAAGQVFHGDIPRYMAGDPTAGKLSGGFLFKMYGLPAAAIAIWHSAKPENRAKVGGIMISAALTSFLTGITEPIEFSFMFVAPILYIIHAILAGLAFPICILLGMRDGTSFSHGLIDFIVLSGNSSKLWLFPIVGIGYAIVYYTIFRVLIKALDLKTPGREDATEDAKATGTSEMAPALVAAFGGKENITNLDACITRLRVSVADVSKVDQAGLKKLGAAGVVVAGSGVQAIFGTKSDNLKTEMDEYIRNH

SEQ ID NO: 16 - DNA, 1434 bp, Artificial Sequence
  WT ptsG
atgtttaagaatgcatttgctaacctgcaaaaggtcggtaaatcgctgatgctgccggtatccgtactgcctatcgcaggtattctgctgggcgtcggttccgcgaatttcagctggctgcccgccgttgtatcgcatgttatggcagaagcaggcggttccgtctttgcaaacatgccactgatttttgcgatcggtgtcgccctcggctttaccaataacgatggcgtatccgcgctggccgcagttgttgcctatggcatcatggttaaaaccatggccgtggttgcgccactggtactgcatttacctgctgaagaaatcgcctctaaacacctggcggatactggcgtactcggagggattatctccggtgcgatcgcagcgtacatgtttaaccgtttctaccgtattaagctgcctgagtatcttggcttctttgccggtaaacgctttgtgccgatcatttctggcctggctgccatctttactggcgttgtgctgtccttcatttggccgccgattggttctgcaatccagaccttctctcagtgggctgcttaccagaacccggtagttgcgtttggcatttacggtttcatcgaacgttgcctggtaccgtttggtctgcaccacatctggaacgtacctttccagatgcagattggtgaatacaccaacgcagcaggtcaggttttccacggcgacattccgcgttatatggcgggtgacccgactgcgggtaaactgtctggtggcttcctgttcaaaatgtacggtctgccagctgccgcaattgctatctggcactctgctaaaccagaaaaccgcgcgaaagtgggcggtattatgatctccgcggcgctgacctcgttcctgaccggtatcaccgagccgatcgagttctccttcatgttcgttgcgccgatcctgtacatcatccacgcgattctggcaggcctggcattcccaatctgtattcttctggggatgcgtgacggtacgtcgttctcgcacggtctgatcgacttcatcgttctgtctggtaacagcagcaaactgtggctgttcccgatcgtcggtatcggttatgcgattgtttactacaccatcttccgcgtgctgattaaagcactggatctgaaaacgccgggtcgtgaagacgcgactgaagatgcaaaagcgacaggtaccagcgaaatggcaccggctctggttgctgcatttggtggtaaagaaaacattactaacctcgacgcatgtattacccgtctgcgcgtcagcgttgctgatgtgtctaaagtggatcaggccggcctgaagaaactgggcgcagcgggcgtagtggttgctggttctggtgttcaggcgattttcggtactaaatccgataacctgaaaaccgagatggatgagtacatccgtaaccactaa

(19) United States
(12) Patent Application Publication
(10) Pub. No.: US 2025/0305032 A1
(43) Pub. Date: Oct. 2, 2025

(54) METHOD FOR PROVIDING RARE SUGAR ASSIMILATION ABILITY DETERMINING GENE CLUSTER OF RARE SUGAR NON-METABOLIC STRAIN

(71) Applicant: INDUSTRY-ACADEMIC COOPERATION FOUNDATION, YONSEI UNIVERSITY, Seoul (KR)

(72) Inventors: Dong Woo LEE, Daegu (KR); Yun Hye JOO, Seoul (KR); Jae Yoon SUNG, Seoul (KR)

(21) Appl. No.: 18/565,775

(22) PCT Filed: Jun. 2, 2022
(86) PCT No.: PCT/KR2022/007860

§ 371 Date: Nov. 30, 2023

(30) Foreign Application Priority Data
Jun. 1, 2021 (KR) 10-2021-0070803
Jun. 14, 2021 (KR) 10-2021-0076938
Jun. 14, 2021 (KR) 10-2021-0076939
Jun. 2, 2022 (KR) 10-2022-0067642
Jun. 2, 2022 (KR) 10-2022-0067643

(51) Int. Cl. C12Q 1/6827; C12N 9/12; C12N 9/88; C12N 15/52

(52) U.S. Cl. CPC C12Q 1/6827; C12N 9/1205; C12N 9/88; C12N 15/52; C12Q 2600/156; C12Y 207/01056; C12Y 401/0204

(57) ABSTRACT

The present invention relates to a method of providing a gene cluster that determines the rare sugar utilization ability of a strain unable to metabolize a rare sugar. A novel sugar metabolic pathway may be constructed using an expression cassette or a vector containing the same according to the present invention, and a mutant strain transformed with the vector or a mutant strain containing a mutated gene has a novel sugar metabolic pathway constructed therein. In addition, the present invention relates to a method for selecting a variant with increased activity, and a composition for selecting a variant with increased activity may be advantageously used to select a variant with increased activity by detecting the mutation of SEQ ID NO: 11.

### CROSS-REFERENCE TO RELATED APPLICATIONS

This application is a 35 U.S.C. § 371 national stage application of PCT International Application No. PCT/KR2022/007860, filed Jun. 2, 2022, which claims the benefit of the filing date of Korean Patent Application No. 10-2021-0070803 filed with the Korean Intellectual Property Office on Jun. 1, 2021, and claims the benefit of the filing date of Korean Patent Application Nos. 10-2021-0076938 and 10-2021-0076939 filed with the Korean Intellectual Property Office on Jun. 14, 2021, and claims the benefit of the filing date of Korean Patent Application Nos. 10-2022-0067642 and 10-2022-0067643 filed with the Korean Intellectual Property Office on Jun. 2, 2022, the entire contents of which are incorporated herein by reference. The above-referenced PCT International Application was published in the Korean language as International Publication No. WO 2022/255823 on Dec. 8, 2022.

### STATEMENT REGARDING ELECTRONIC FILING OF A SEQUENCE LISTING

A Sequence Listing in ASCII text format, submitted under 37 C.F.R. § 1.821, entitled 1433-67_ST25.txt, 35,196 bytes in size, generated on Jun. 21, 2024 and filed electronically, is provided in lieu of a paper copy. This Sequence Listing is hereby incorporated herein by reference into the specification for its disclosures.

### TECHNICAL FIELD

The present invention relates to a method of providing a gene cluster that determines the rare sugar utilization ability of a strain unable to metabolize a rare sugar.

The present invention also relates to a strain in which the fructose metabolic gene has been mutated.

Moreover, the present invention relates to a method for selecting variants with increased activity.

### BACKGROUND ART

D-tagatose is an isomer of D-galactose and is a natural sugar present in fruits, milk, cheese, etc. Because D-tagatose has various health functional properties and a sweet taste very similar to sugar, it is used as an alternative sweetener that can satisfy both health and taste when applied to various products.

Meanwhile, directed evolution technology is generally used as an improved technology to change the properties of enzymes to suit the desired purposes, such as enhancing the activity and structural stability of the enzyme or imparting activity against new substrates. The most commonly used method to generate mutant libraries for performing this technology is the error-prone PCR method, which is a method of randomly introducing mutations by controlling the error rate of DNA polymerase during PCR. After expressing the protein using the mutant strains created in this way, an improved enzyme with excellent activity is obtained by selecting a mutant strain with good activity. Developing an efficient screening technology suitable for the characteristics and purpose of a desired enzyme can be considered as a key technology of directed evolution.

Korean Patent Application Publication No. 10-2018-0074550 discloses a recombinant strain with a modified sugar metabolic pathway, which is obtained by introducing into a strain an enzyme from another strain, and the possibility of obtaining D-tagatose by the same. However, recently, profitability has decreased due to the rise in raw material prices, and thus there is an increasing need to develop a strain that is capable of producing D-tagatose through a novel metabolic pathway.

Accordingly, the present inventors have conducted studies and identified a mutant strain having a modified sugar metabolic pathway and the use thereof, thereby completing the present invention.

In addition, in order to produce tagatose having this functionality, attempts have been made to remodel the tagatose production pathway in various ways, such as adding mutations to the enzymes of strains or introducing genes from other strains. However, since these attempts have been made to identify only the possibility of individual tagatose production, no substantial studies have been conducted on comparing metabolic ability with other strains or predicting the selection of sugar metabolic ability of a strain of interest.

Accordingly, the present inventors have studied a method capable of selecting a strain having improved sugar metabolism ability, thereby completing the present invention.

### DISCLOSURE

### Technical Problem

An object of an aspect of the present invention is to provide an expression cassette comprising a gene encoding a novel fructose-1-phosphate kinase in which alanine (A) at position 39 in the amino acid sequence of SEQ ID NO: 1 is substituted with serine (S).

An object of another aspect of the present invention is to provide a recombinant vector containing the expression cassette.

An object of still another aspect of the present invention is to provide a mutant strain transformed with the recombinant vector, and thereby to provide a strain able to metabolize D-tagatose from a strain unable to metabolize D-tagatose.

An object of yet another aspect of the present invention is to provide a mutant strain comprising any one or more gene sequences selected from among 1) the gene sequence of SEQ ID NO: 2 encoding a novel fructose-1-phosphate kinase; 2) deletion of the gene sequence of SEQ ID NO: 3, which is a Cra-binding site; and 3) the gene sequence of SEQ ID NO: 4.

An object of a further aspect of the present invention is to provide a method for producing a strain able to metabolize D-tagatose, the method comprising a step of culturing the mutant strain in a medium containing D-tagatose.

An object of an aspect of the present invention is to provide an expression cassette comprising: a gene mutated to inactivate the fructose-bisphosphate aldolase class 2 (FbaA) of SEQ ID NO: 5; a gene encoding a novel phosphotransferase system G(PstG) in which valine (V) at position 12 in the amino acid sequence of SEQ ID NO: 6 is substituted with phenylalanine (F); and a gene mutated to inactivate the putative aga operon transcriptional repressor (AgaR) of SEQ ID NO: 7.

An object of another aspect of the present invention is to provide a recombinant vector containing the expression cassette.

An object of still another aspect of the present invention is to provide a mutant strain transformed with the recombinant vector.

An object of yet another aspect of the present invention is to provide a mutant strain comprising any one or more of 1) a gene mutated to inactivate the fructose-bisphosphate aldolase class 2 (FbaA) of SEQ ID NO: 5; 2) a gene encoding a novel phosphotransferase system G in which valine (V) at position 12 in the amino acid sequence of SEQ ID NO: 6 is substituted with phenylalanine (F); and 3) a gene mutated to inactivate the putative aga operon transcriptional repressor (AgaR) of SEQ ID NO: 7.

An object of still yet another aspect of the present invention is to provide a method for producing a strain unable to metabolize D-fructose while being able to metabolize D-tagatose, the method comprising a step of culturing the mutant strain in a medium containing D-fructose or D-tagatose.

In accordance with one embodiment, there is provided a composition for selecting a variant with increased activity, the composition comprising an agent capable of detecting mutation at any one or more of positions 16, 92, 95, 105, 129, 148, 193, 236, 324, 341 and 362 in the amino acid sequence of SEQ ID NO: 11.

In accordance with another embodiment, there is provided a kit for selecting a variant with increased activity, the kit comprising the composition.

In accordance with still another embodiment, there is provided a method of providing information for selecting a variant with increased activity, the method comprising steps of: detecting mutation from a sample using the composition; and checking, based on the detected mutation, whether the sample is a variant with increased activity.

### Technical Solution

An aspect of the present invention provides an expression cassette comprising a gene encoding a novel fructose-1-phosphate kinase in which alanine (A) at position 39 in the amino acid sequence of SEQ ID NO: 1 is substituted with serine (S).

In one embodiment of the present invention, the gene encoding the novel fructose-1-phosphate kinase may be SEQ ID NO: 2.

In one embodiment of the present invention, the expression cassette may be one in which a Cra-binding site is deleted.

In one embodiment of the present invention, the deletion of the Cra-binding site may correspond to deletion of the sequence of SEQ ID NO: 3 in the expression cassette.

In one embodiment of the present invention, the expression cassette may further comprise a mutation sequence between a sequence encoding LacI and a sequence encoding T7 RNAP.

In one embodiment of the present invention, the mutation sequence between the sequence encoding LacI and the sequence encoding T7 RNAP may be mutation in a T7 RNAP core promoter region.

In one embodiment of the present invention, the mutation sequence between the sequence encoding LacI and the sequence encoding T7 RNAP may be SEQ ID NO: 4.

Another aspect of the present invention provides a recombinant vector containing the expression cassette.

Still another aspect of the present invention provides a mutant strain transformed with the recombinant vector.

Yet another aspect of the present invention provides a mutant strain comprising any one or more gene mutation sequences selected from among 1) the gene mutation sequence of SEQ ID NO: 2 encoding a novel fructose-1-phosphate kinase, 2) deletion of the gene sequence of SEQ ID NO: 3, which is a Cra-binding site, and 3) the gene mutation sequence of SEQ ID NO: 4.

In one embodiment of the present invention, the recombinant strain may be E. coli.

In one embodiment of the present invention, the recombinant strain may be able to metabolize D-tagatose.

A still yet another aspect of the present invention provides a method for producing a strain able to metabolize D-tagatose, the method comprising a step of culturing the mutant strain in a medium containing D-tagatose.

An aspect of the present invention provides an expression cassette comprising: a gene mutated to inactivate the fructose-bisphosphate aldolase class 2 (FbaA) of SEQ ID NO: 5; a gene encoding a novel phosphotransferase system G in which valine (V) at position 12 in the amino acid sequence of SEQ ID NO: 6 is substituted with phenylalanine (F); and a gene mutated to inactivate the putative aga operon transcriptional repressor (AgaR) of SEQ ID NO: 7.

In one embodiment of the present invention, the gene encoding the novel phosphotransferase system G may be SEQ ID NO: 9.

In one embodiment of the present invention, the inactivation of the putative aga operon transcriptional repressor may cause tagatose aldolase (KbaY) to be expressed.

In one embodiment of the present invention, the inactivation may be achieved by gene deletion.

Another aspect of the present invention provides a recombinant vector containing the expression cassette, and a mutant strain transformed with the recombinant vector.

Still another aspect of the present invention provides a mutant strain comprising any one or more of 1) a gene mutated to inactivate the fructose-bisphosphate aldolase class 2 (FbaA) of SEQ ID NO: 5; 2) a gene encoding a novel phosphotransferase system G in which valine (V) at position 12 in the amino acid sequence of SEQ ID NO: 6 is substituted with phenylalanine (F); and 3) a gene mutated to inactivate the putative aga operon transcriptional repressor (AgaR) of SEQ ID NO: 7.

In one embodiment of the present invention, the recombinant strain may be E. coli.

In one embodiment of the present invention, the recombinant strain may be a mutant strain that is able to metabolize D-tagatose while being unable or having reduced ability to metabolize D-fructose.

Yet another aspect of the present invention provides a method for producing a strain unable to metabolize D-fructose while being able to metabolize D-tagatose, the method comprising a step of culturing the mutant strain in a medium containing D-fructose or D-tagatose.

An aspect of the present invention provides a composition for selecting a variant with increased activity, the composition comprising an agent capable of detecting mutation at any one or more of positions 16, 92, 95, 105, 129, 148, 193, 236, 324, 341 and 362 in the amino acid sequence of SEQ ID NO: 11.

In one embodiment of the present invention, the mutation may be mutation at any one or more positions selected from among V16, R92, F95, T105, N129, F148, K193, R236, K324, H341, and H362.

In one embodiment of the present invention, the mutation may be any one or more of V16A, R92S, F95I, T105A, N129Y, F148S, K193E, R236S, K324N, H341L, and H362I.

In one embodiment of the present invention, the agent may be used in any one of polymerase chain reaction, reverse transcription-polymerase chain reaction (RT-PCR), competitive reverse transcription-polymerase chain reaction (competitive RT-PCR), RNase protection assay (RNase, S1 nuclease assay), in situ hybridization, nucleic acid microarray assay, next-generation sequencing, and Northern blotting.

Another aspect of the present invention provides a kit for selecting a variant with increased activity, the kit comprising the composition.

Still another aspect of the present invention provides a method of providing information for selecting a variant with increased activity, the method comprising steps of: detecting mutation from a sample using the composition; and checking, based on the detected mutation, whether the sample is a variant with increased activity.

### Advantageous Effects

A novel sugar metabolic pathway may be constructed using the expression cassette or the vector containing the same according to the present invention, and a mutant strain transformed with the vector or a mutant strain containing a mutated gene has a novel sugar metabolic pathway constructed therein.

In addition, the composition for selecting a variant with increased activity according to the present invention may be advantageously used to select a variant with increased activity by detecting the mutation of SEQ ID NO: 11.

### BRIEF DESCRIPTION OF DRAWINGS

FIG. 1 shows a process of constructing a mutant strain of the present invention. Specifically, FIG. 1a shows the results of comparing the gene clusters between four strains (S. enterica, K. pneumociae, K. oxytoca, and B. licheniformis) capable of tagatose utilization and E. coli, FIG. 1b shows the results of identifying mutation sites by comparing a parent strain (E. coli BL12 (DE3)) with a constructed mutant strain (pET28a-gatY/E. coli BL21(DE3)), and FIG. 1c is a graph of examining the growth rate of the constructed mutant strain depending on subculture.

FIG. 2 shows the results of analyzing the activity of the mutant strain. Specifically, FIG. 2a shows the results of comparing the kinase activities of a WT strain and the mutant strain, FIG. 2b shows the results of analyzing the mRNA expression levels of genes in media containing three sugar sources (Glc (glucose), Fru (fructose), and Tag (tagatose)) for the mutant strain by qRT-PCR, and FIG. 2c shows tagatose-metabolizing activity introduced through mutation.

FIG. 3 shows the results of examining the growth of the mutant strain of the present invention. Specifically, FIG. 3a shows the results of examining the growth profile of a mutant strain in which the fruK_A39S mutation was introduced and the Cra gene-binding site was deleted (a strain in which two sites were mutated), and FIG. 3b shows the results of examining the growth profile of a mutant strain in which the fruK_A39S mutation was introduced, the Cra gene-binding site was deleted and the T7RNAP core promoter region was mutated (a strain in which three sites were mutated).

FIG. 4 shows a process of constructing the mutant strain of the present invention.

FIG. 5 shows a novel sugar metabolic pathway constructed through the expression vector or mutations of the present invention.

FIG. 6 shows the operon expression mechanism according to the AgaR mutation of the present invention.

FIG. 7 shows the results of analyzing the activity of a strain containing a mutation.

FIG. 8 shows the growth profile of the strain of the present invention during culture. Specifically, the left panel shows the growth profile of the strain during culture depending on a carbon source, and the right panel shows the growth profile of the strain when the strain was cultured with fructose epimerase.

FIG. 9 shows a mutant library construction process.

FIG. 10 shows the results of comparing the activity levels between the mutant libraries of the present invention.

FIG. 11 shows the results of evaluating the activity of the variant by structural prediction according to the mutations of the present invention.

### BEST MODE

An aspect of the present invention provides an expression cassette comprising a gene encoding a novel fructose-1-phosphate kinase in which alanine (A) at position 39 in the amino acid sequence of SEQ ID NO: 1 is substituted with serine (S).

The amino acid sequence of SEQ ID NO: 1 refers to the amino acid sequence of wild-type fructose-1-phosphate kinase (fruK). In the present invention, it is possible to metabolize D-tagatose instead of D-fructose through the novel fructose-1-phosphate kinase in which alanine (A) at position 39 in the amino acid sequence of SEQ ID NO: 1 is substituted with serine (S).

The fructose-1-phosphate kinase (fruK), also called 1-phosphofructokinase, is an in vivo enzyme that functions to convert fructose-1-phosphate and ATP in vivo into fructose-1,6-diphosphate and ADP.

In one embodiment of the present invention, the gene encoding the fructose-1-phosphate kinase may be SEQ ID NO: 2.

TABLE 1

| Sequence | |
|---|---|
| name | Sequence |
| Amino acid | MSRRVATITLNPAYDLVGFCPEIERGEVNLVKTTGLHAAGKGINVAKVLKDLGIDVTVGG |
| sequence of | FLGKDNQDGFQQLFSELGIANRFQVVQGRTRINVKLTEKDGEVTDFNFSGFEVTPADWER |
| E.coli | FVTDSLSWLGQFDMVCVSGSLPSGVSPEAFTDWMTRLRSQCPCIIFDSSREALVAGLKAA |
| BL21 (DE3) | PWLVKPNRRELEIWAGRKLPEMKDVIEAAHALREQGIAHVVISLGAEGALWVNASGEWIA |
| Fruk WT | KPPSVDVVSTVGAGDSMVGGLIYGLLMRESSEHTLRLATAVAALAVSQSNVGITDRPQLA |
| (SEQ ID NO: | AMMARVDLQPEN |
| 1) | |
| Amino acid | MSRRVATITLNPAYDLVGFCPEIERGEVNLVKTTGLHASGKGINVAKVLKDLGIDVTVGG |
| sequence of | FLGKDNQDGFQQLFSELGIANRFQVVQGRTRINVKLTEKDGEVTDENFSGFEVTPADWER |
| E.coli | FVTDSLSWLGQFDMVCVSGSLPSGVSPEAFTDWMTRLRSQCPCIIFDSSREALVAGLKAA |
| BL21 (DE3) | PWLVKPNRRELEIWAGRKLPEMKDVIEAAHALREQGIAHVVISLGAEGALWVNASGEWIA |
| Fruk A39S | KPPSVDVVSTVGAGDSMVGGLIYGLLMRESSEHTLRLATAVAALAVSQSNVGITDRPQLA |
| mutant (SEQ | AMMARVDLQPEN |
| ID NO : 12) | |
| Nucleotide | ATGAGCAGACGTGTTGCTACTATCACCCTTAATCCGGCTTATGACCTTGTTGGTTTCTGC |
| sequence of | CCGGAAATTGAACGCGGCGAAGTGAACCTGGTGAAAACCACCGGTCTGCATGCGGCGGGT |
| Fruk WT | AAAGGCATCAACGTGGCCAAAGTATTAAAAGACCTGGGAATTGATGTCACCGTTGGCGGC |
| (SEQ ID | TTCCTCGGTAAAGACAATCAGGATGGTTTTCAGCAACTGTTCAGCGAGCTGGGCATTGCC |
| NO:13) | AACCGTTTCCAGGTTGTACAGGGGCGCACTCGAATTAACGTTAAGCTGACGGAAAAAGAC |
| | GGCGAAGTGACCGACTTCAACTTCTCGGGTTTTGAAGTCACCCCCGCCGACTGGGAACGC |
| | TTTGTGACTGATTCTCTGAGCTGGCTCGGTCAGTTCGATATGGTCTGTGTCAGCGGAAGC |
| | TTACCGTCAGGCGTCAGCCCGGAAGCGTTCACCGACTGGATGACTCGCCTGCGTAGTCAG |
| | TGTCCTTGCATTATCTTTGATAGTAGCCGTGAAGCGTTAGTAGCAGGTTTGAAAGCGGCA |
| | CCGTGGCTGGTGAAACCTAACCGCCGCGAGCTGGAAATCTGGGCAGGCCGTAAACTGCCT |
| | GAAATGAAAGATGTGATTGAAGCTGCGCATGCGCTGCGTGAACAAGGTATCGCGCATGTT |
| | GTTATTTCACTGGGTGCCGAAGGCGCGCTTTGGGTTAATGCCTCCGGCGAATGGATCGCC |
| | AAACCACCGTCAGTCGATGTCGTAAGCACCGTTGGCGCAGGGGATTCTATGGTTGGTGGC |
| | CTGATTTATGGCTTGCTGATGCGTGAATCCAGTGAACACACACTGCGTCTGGCGACAGCT |
| | GTTGCAGCCCTGGCGGTAAGTCAAAGCAATGTGGGTATTACCGATCGTCCGCAGTTGGCC |
| | GCAATGATGGCGCGCGTCGACTTACAACCTTTTAACTGA |
| Nucleotide | ATGAGCAGACGTGTTGCTACTATCACCCTTAATCCGGCTTATGACCTTGTTGGTTTCTGC |
| sequence of | CCGGAAATTGAACGCGGCGAAGTGAACCTGGTGAAAACCACCGGTCTGCATGCGTCGGGT |
| mutant Fruk | AAAGGCATCAACGTGGCCAAAGTATTAAAAGACCTGGGAATTGATGTCACCGTTGGCGGC |
| A39S (SEQ | TTCCTCGGTAAAGACAATCAGGATGGTTTTCAGCAACTGTTCAGCGAGCTGGGCATTGCC |
| ID NO: 2) | AACCGTTTCCAGGTTGTACAGGGGCGCACTCGAATTAACGTTAAGCTGACGGAAAAAGAC |
| | GGCGAAGTGACCGACTTCAACTTCTCGGGTTTTGAAGTCACCCCCGCCGACTGGGAACGC |
| | TTTGTGACTGATTCTCTGAGCTGGCTCGGTCAGTTCGATATGGTCTGTGTCAGCGGAAGC |
| | TTACCGTCAGGCGTCAGCCCGGAAGCGTTCACCGACTGGATGACTCGCCTGCGTAGTCAG |
| | TGTCCTTGCATTATCTTTGATAGTAGCCGTGAAGCGTTAGTAGCAGGTTTGAAAGCGGCA |
| | CCGTGGCTGGTGAAACCTAACCGCCGCGAGCTGGAAATCTGGGCAGGCCGTAAACTGCCT |
| | GAAATGAAAGATGTGATTGAAGCTGCGCATGCGCTGCGTGAACAAGGTATCGCGCATGTT |
| | GTTATTTCACTGGGTGCCGAAGGCGCGCTTTGGGTTAATGCCTCCGGCGAATGGATCGCC |
| | AAACCACCGTCAGTCGATGTCGTAAGCACCGTTGGCGCAGGGGATTCTATGGTTGGTGGC |
| | CTGATTTATGGCTTGCTGATGCGTGAATCCAGTGAACACACACTGCGTCTGGCGACAGCT |
| | GTTGCAGCCCTGGCGGTAAGTCAAAGCAATGTGGGTATTACCGATCGTCCGCAGTTGGCC |
| | GCAATGATGGCGCGCGTCGACTTACAACCTTTTAACTGA |

In one embodiment of the present invention, the expression cassette may be one in which a Cra-binding site is deleted, and the deletion of the Cra-binding site may correspond to deletion of the sequence of SEQ ID NO: 3 in the expression cassette.

TABLE 2

| Sequence name | Sequence |
|---|---|
| Nucleotide | Tgaaacgatt cagcctctat |
| sequence of | gagaaaaaaa gcgccaacct |
| Cra-binding | ggcttagggt taaagacaag |
| site deleted | atcgcgc |
| (SEQ ID NO: 3) | |

The Cra (catabolite repressor/activator) is induced by the fructose-1-phosphate and fructose-1,6-biphosphate, and if there is a Cra-binding site in the expression cassette, expression of the gene encoding the novel fructose-1-phosphate kinase may be limited, but the expression level of the gene encoding the novel fructose-1-phosphate kinase may be increased by deleting this Cra-binding site.

In one embodiment of the present invention, the expression cassette may further comprise a mutation sequence between a sequence encoding LacI and a sequence encoding T7 RNAP. More specifically, the mutation sequence between the sequence encoding LacI and the sequence encoding T7 RNAP may be a T7 RNAP core promoter region. More specifically, the mutation sequence between the sequence encoding LacI and the sequence encoding T7 RNAP may be SEQ ID NO: 4.

TABLE 3

| Sequence | |
|---|---|
| name | Sequence |
| Wt T7RNAP | gcaaaccgcctctccccgcgcgttggccga |
| core | ttcattaatgcagctggcacgacaggtttc |
| promoter | ccgactggaaagcgggcagtgagcgcaacg |
| region (SEQ | caattaatgtaagttagctcactcattagg |
| ID NO: 14) | caccccaggctttacactttatgcttccgg |
| | ctcgtataatgtgtggaattgtgagcggat |
| | aacaatttcacacaggaaacagctatgacc |
| | atgattacggattcactggccgtcgtttta |
| | caacgtcgtgactgggaaaaccctggcgtt |
| | acccaactt |
| Mutated | gcaaaccgcctctccccgcgcgttggccga |
| T7RNAP core | ttcattaatgcagctggcacgacaggtttc |
| promoter | ccgactggaaagcgggcagtgagcgcaacg |
| region (SEQ | caattaatgtaagttagctcactcattagg |
| ID NO: 4) | caccccaggctttacactttatgcttccgg |
| | ctcgtatgttgtgtgaaattgtgagcggat |
| | aacaatttcacacaggaaacagctatgacc |
| | atgattacggattcactggccgtcgtttta |
| | caacgtcgtgactgggaaaaccctggcgtt |
| | acccaactt |

The LacI refers to a gene encoding a lac repressor (LacI), which is a DNA-binding protein that inhibits the expression of genes encoding proteins participating in lactose metabolism in microorganisms.

The T7 RNAP refers to an RNA polymerase derived from T7 bacteriophage that catalyzes the formation of RNA from DNA in the 5′→3′ direction (EC:2.7.7.).

Another aspect of the present invention provides a recombinant vector containing the expression cassette.

As used herein, the term “recombinant vector” refers to a recombinant DNA molecule containing a desired coding sequence and appropriate nucleic acid sequences necessary for expression of the operably linked coding sequence in a particular host cell. The promoters, enhancers, termination signals and polyadenylation signals available in eukaryotic cells are known.

As used herein, the term “operably linked” refers to a functional linkage between a gene expression regulatory sequence and another nucleotide sequence. The gene expression regulatory sequence may be one or more selected from the group consisting of an origin of replication, a promoter, and a transcription termination sequence (terminator). The transcription termination sequence may be a polyadenylation sequence (pA), and the origin of replication may be an f1 origin of replication, an SV40 origin of replication, a pMB1 origin of replication, an adeno origin of replication, an AAV origin of replication, or a BBV origin of replication, without being limited thereto.

The recombinant vector according to one embodiment of the present invention may be selected from the group consisting of plasmid vectors, cosmid vectors, and viral vectors such as bacteriophage vectors, adenovirus vectors, retrovirus vectors, and adeno-associated virus vectors. Vectors that may be used as recombinant expression vectors may be constructed based on plasmids (e.g., pcDNA series, pSC101, pGV1106, pACYC177, ColE1, pKT230, pME290, pBR322, pUC8/9, pUC6, pBD9, pHC79, pIJ61, pLAFR1, pHV14, pGEX series, pET series, pUC19, etc.), phages (e.g., λgt4λB, λ-Charon, λΔz1, M13, etc.), or viral vectors (e.g., adeno-associated virus (AAV) vector, etc.), etc., which are used in the art, without being limited thereto.

The recombinant vector of the present invention may further contain one or more selectable markers. The markers are nucleic acid sequences that have the properties that can generally be selected by chemical methods, and include all genes that can distinguish transfected cells from non-transfected cells. Examples of the markers include, but are not limited to, genes resistant to herbicides such as glyphosate, glufosinate ammonium, or phosphinothricin, and genes resistant to antibiotics such as ampicillin, kanamycin, G418, bleomycin, hygromycin, or chloramphenicol.

The recombinant vector of the present invention may be constructed using genetic recombination technology well known in the art, and site-specific DNA cleavage and ligation may be performed using enzymes generally known in the art.

Another aspect of the present invention provides a mutant strain transformed with the above-described recombinant vector.

For transformation with the recombinant vector, a transfer method widely known in the art may be used. For example, when the host cell is a prokaryotic cell, the transfer may be performed using a CaCl₂ method or an electroporation method, and when the host cell is a eukaryotic cell, the transfer may be performed using a microinjection, calcium phosphate precipitation, electroporation, liposome-mediated transfection, heat shock, or gene bombardment method, without being limited thereto.

Another aspect of the present invention provides a mutant strain comprising any one or more gene sequences selected from among 1) the gene sequence of SEQ ID NO: 2 encoding a novel fructose-1-phosphate kinase, 2) deletion of the gene sequence of SEQ ID NO: 3, which is a Cra-binding site, and 3) the gene mutation sequence of SEQ ID NO: 4.

The mutant strain may comprise, in addition to the mutant strain transformed with the above-described recombinant vector, any one or more gene mutation sequences selected from among 1) the gene mutation sequence of SEQ ID NO: 2 encoding a novel fructose-1-phosphate kinase, 2) deletion of the gene sequence of SEQ ID NO: 3, which is a Cra-binding site, and 3) the gene sequence of SEQ ID NO: 4, which are introduced by various mutagenesis methods.

The gene sequence included in the mutant strain may comprise any one gene sequence selected from among 1) the gene mutation sequence of SEQ ID NO: 2 encoding a novel fructose-1-phosphate kinase, 2) deletion of the gene sequence of SEQ ID NO: 3, which is a Cra-binding site, and 3) the gene mutation sequence of SEQ ID NO: 4; or
comprise 1) the gene mutation sequence of SEQ ID NO: 2 encoding a novel fructose-1-phosphate kinase, and 2) deletion of the gene sequence of SEQ ID NO: 3, which is a Cra-binding site; or comprise 1) the gene mutation sequence of SEQ ID NO: 2 encoding a novel fructose-1-phosphate kinase, and 3) the gene mutation sequence of SEQ ID NO: 4; or 2) deletion of the gene sequence of SEQ ID NO: 3, which is a Cra-binding site, and 3) the gene mutation sequence of SEQ ID NO: 4; or
comprise 1) the gene sequence of SEQ ID NO: 2 encoding a novel fructose-1-phosphate kinase, 2) the deletion sequence of SEQ ID NO: 4 in the Cra-binding site of SEQ ID NO: 3, and 3) the gene mutation sequence of SEQ ID NO: 4.

The host of the mutant strain may be any host known in the art. Prokaryotic cells usable as the host include, for example, E. coli JM109, E. coli BL21, E. coli RR1, E. coli LE392, E. coli B, E. coli X 1776, E. coli W3110, Bacillus sp. strains such as Bacillus subtilis and Bacillus thuringiensis, and Enterobacteriaceae strains such as Salmonella typhimurium, Serratia marcescens and various Pseudomonas species. Examples of host cells that may be used for transformation into eukaryotic cells include yeast (Saccharomyces cerevisiae), insect cells, plant cells, and animal cells such as SP2/0, CHO (Chinese hamster ovary) K1, CHO DG44, PER.C6, W138, BHK, COS-7, 293, HepG2, Huh7, 3T3, RIN and MDCK cell lines. Specifically, the host of the mutant strain may be E. coli.

In one embodiment of the present invention, the mutant strain is able to metabolize D-tagatose. Specifically, the recombinant strain is able to metabolize D-tagatose because it contains the above-described expression cassette or mutations. The phrase “able to metabolize D-tagatose” means utilizing D-tagatose as an energy source.

Another aspect of the present invention provides a method for producing a strain able to metabolize D-tagatose, the method comprising a step of culturing the mutant strain in a medium containing D-tagatose.

The step of culturing is a step of culturing the mutant strain in a medium containing D-tagatose, wherein the medium may contain known components for culturing the mutant strain.

An aspect of the present invention provides an expression cassette comprising: a gene mutated to inactivate the fructose-bisphosphate aldolase class 2 (FbaA) of SEQ ID NO: 5; a gene encoding a novel phosphotransferase system G in which valine (V) at position 12 in the amino acid sequence of SEQ ID NO: 6 is substituted with phenylalanine (F); and a gene mutated to inactivate the putative aga operon transcriptional repressor (AgaR) of SEQ ID NO: 7.

The amino acid sequence of SEQ ID NO: 5 refers to the amino acid sequence of wild-type fructose-bisphosphate aldolase class 2 (FbaA). In the present invention, the gene encoding the amino acid sequence of wild-type fructose-bisphosphate aldolase class 2 (FbaA) of SEQ ID NO: 5 may be mutated to be inactivated, thereby inhibiting fructose utilization and improving tagatose utilization.

The fructose-bisphosphate aldolase class 2 (FbaA) functions to catalyze the aldol condensation of dihydroxyacetone phosphate (DHAP or glycerone-phosphate) with glyceraldehyde 3-phosphate (G3P) to form fructose 1,5-biphosphate (FBP) in gluconeogenesis and the reverse reaction in glycolysis.

In one embodiment of the present invention, the gene encoding fructose-bisphosphate aldolase class 2 (FbaA) of SEQ ID NO: 5 may comprise SEQ ID NO: 8.

TABLE 4

| Sequence | | SEQ ID |
|---|---|---|
| name | Sequence | NO. |
| Amino acid | MSKIFDFVKP GVITGDDVQK VFQVAKENNF ALPAVNCVGT | 5 |
| sequence | DSINAVLETA | |
| of FbaA WT | AKVKAPVIVQ FSNGGASFIA GKGVKSDVPQ GAAILGAISG | |
| | AHHVHQMAEH | |
| | YGVPVILHTD HCAKKLLPWI DGLLDAGEKH FAATGKPLES | |
| | SHMIDLSEES | |
| | LQENIEICSK YLERMSKIGM TLEIELGCTG GEEDGVDNSH | |
| | MDASALYTQP | |
| | EDVDYAYTEL SKISPRFTIA ASFGNVHGVY KPGNVVLTPT | |
| | ILRDSQEYVS | |
| | KKHNLPHNSL NFVFHGGSGS TAQEIKDSVS YGVVKMNIDT | |
| | DTQWATWEGV | |
| | LNYYKANEAY LQGQLGNPKG EDQPNKKYYD PRVWLRAGQT | |
| | SMIARLEKAF | |
| | QELNAIDVL | |
| Gene | atgtctaaga tttttgattt cgtaaaacct ggcgtaatca | 8 |
| sequence | ctggtgatga cgtacagaaa gttttccagg tagcaaaaga | |
| of FbaA WT | aaacaacttc gcactgccag cagtaaactg cgtcggtact | |
| | gactccatca acgccgtact ggaaaccgct gctaaagtta | |
| | aagcgccggt tatcgttcag | |
| | ttctccaacg gtggtgcttc ctttatcgct ggtaaaggcg | |
| | tgaaatctga cgttccgcag | |
| | ggtgctgcta tcctgggcgc gatctctggt gcgcatcacg | |
| | ttcaccagat ggctgaacat | |
| | tatggtgttc cggttatcct gcacactgac cactgcgcga | |
| | agaaactgct gccgtggatc | |
| | gacggtctgt tggacgcggg tgaaaaacac ttcgcagcta | |
| | ccggtaagcc gctgttctct | |
| | tctcacatga tcgacctgtc tgaagaatct ctgcaagaga | |
| | acatcgaaat ctgctctaaa | |
| | tacctggagc gcatgtccaa aatcggcatg actctggaaa | |
| | tcgaactggg ttgcaccggt | |
| | ggtgaagaag acggcgtgga caacagccac atggacgctt | |
| | ctgcactgta cacccagccg | |
| | gaagacgttg attacgcata caccgaactg agcaaaatca | |
| | gcccgcgttt caccatcgca | |
| | gcgtccttcg gtaacgtaca cggtgtttac aagccgggta | |
| | acgtggttct gactccgacc | |
| | atcctgcgtg attctcagga atatgtttcc aagaaacaca | |
| | acctgccgca caacagcctg | |
| | aacttcgtat tccacggtgg ttccggttct actgctcagg | |
| | aaatcaaaga ctccgtaagc | |
| | tacggcgtag taaaaatgaa catcgatacc gatacccaat | |
| | gggcaacctg ggaaggcgtt | |
| | ctgaactact acaaagcgaa cgaagcttat ctgcagggtc | |
| | agctgggtaa cccgaaaggc | |
| | gaagatcagc cgaacaagaa atactacgat ccgcgcgtat | |
| | ggctgcgtgc cggtcagact | |
| | tcgatgatcg ctcgtctgga gaaagcattc caggaactga | |
| | acgcgatcga cgttctgtaa | |

The amino acid sequence of SEQ ID NO: 6 refers to the amino acid sequence of wild-type phosphotransferase system G (PTS system glucose-specific EIICB component, ptsG). In the present invention, valine (V) at position 12 in the amino acid sequence of SEQ ID NO: 6 is substituted with phenylalanine (F) so that the wild-type ptsG, a glucose transporter, may be mutated and non-phosphorylated fructose may be introduced into a cell.

The phosphotransferase system G (PTS system glucose-specific EIICB component, ptsG) is a major carbohydrate active-transport system and catalyzes the phosphorylation of incoming sugar substrates concomitantly with their translocation across the cell membrane.

In one embodiment of the present invention, the gene encoding the novel phosphotransferase system G may comprise SEQ ID NO: 9.

TABLE 5

| Sequence | | SEQ ID |
|---|---|---|
| name | Sequence | NO. |
| Amino | MFKNAFANLQ KVGKSLMLPV SVLPIAGILL GVGSANFSWL PAVVSHVMAE | 6 |
| acid | AGGSVFANMP LIFAIGVALG FTNNDGVSAL AAVVAYGIMV KTMAVVAPLV | |
| sequence | LHLPAEEIAS KHLADTGVLG GIISGAIAAY MENRFYRIKL PEYLGFFAGK | |
| of ptsG | RFVPIISGLA AIFTGVVLSF IWPPIGSAIQ TFSQWAAYQN PVVAFGIYGE | |
| WT | IERCLVPFGL HHIWNVPFQM QIGEYTNAAG QVFHGDIPRY MAGDPTAGKL | |
| | SGGFLFKMYG LPAAAIAIWH SAKPENRAKV GGIMISAALT SFLTGITEPI | |
| | EFSFMFVAPI LYIIHAILAG LAFPICILLG MRDGTSFSHG LIDFIVLSGN | |
| | SSKLWLFPIV GIGYAIVYYT IFRVLIKALD LKTPGREDAT EDAKATGTSE | |
| | MAPALVAAFG GKENITNLDA CITRLRVSVA DVSKVDQAGL KKLGAAGVVV | |
| | AGSGVQAIFG TKSDNLKTEM DEYIRNH | |
| Amino | MFKNAFANLQ KFGKSLMLPV SVLPIAGILL GVGSANFSWL PAVVSHVMAE | 15 |
| acid | AGGSVFANMP LIFAIGVALG FTNNDGVSAL AAVVAYGIMV KTMAVVAPLV | |
| sequence | LHLPAEEIAS KHLADTGVLG GIISGAIAAY MENRFYRIKL PEYLGFFAGK | |
| of | RFVPIISGLA AIFTGVVLSF IWPPIGSAIQ TFSQWAAYQN PVVAFGIYGF | |
| ptsGV12F | IERCLVPFGL HHIWNVPFQM QIGEYTNAAG QVFHGDIPRY MAGDPTAGKL | |
| | SGGFLFKMYG LPAAAIAIWH SAKPENRAKV GGIMISAALT SFLTGITEPI | |
| | EFSFMEVAPI LYIIHAILAG LAFPICILLG MRDGTSFSHG LIDFIVLSGN | |
| | SSKLWLFPIV GIGYAIVYYT IFRVLIKALD LKTPGREDAT EDAKATGTSE | |
| | MAPALVAAFG GKENITNLDA CITRLRVSVA DVSKVDQAGL KKLGAAGVVV | |
| | AGSGVQAIFG TKSDNLKTEM DEYIRNH | |
| Gene | atgtttaaga atgcatttgc taacctgcaa aaggtcggta aatcgctgat | 16 |
| sequence | gctgccggtatccgtactgc ctatcgcagg tattctgctg ggcgtcggtt | |
| of ptsG | ccgcgaattt cagctggctg | |
| WT | cccgccgttg tatcgcatgt tatggcagaa gcaggcggtt ccgtctttgc | |
| | aaacatgcca | |
| | ctgatttttg cgatcggtgt cgccctcggc tttaccaata acgatggcgt | |
| | atccgcgctg | |
| | gccgcagttg ttgcctatgg catcatggtt aaaaccatgg ccgtggttgc | |
| | gccactggta | |
| | ctgcatttac ctgctgaaga aatcgcctct aaacacctgg cggatactgg | |
| | cgtactcgga | |
| | gggattatct ccggtgcgat cgcagcgtac atgtttaacc gtttctaccg | |
| | tattaagctg | |
| | cctgagtatc ttggcttctt tgccggtaaa cgctttgtgc cgatcatttc | |
| | tggcctggct | |
| | gccatcttta ctggcgttgt gctgtccttc atttggccgc cgattggttc | |
| | tgcaatccag | |
| | accttctctc agtgggctgc ttaccagaac ccggtagttg cgtttggcat | |
| | ttacggtttc | |
| | atcgaacgtt gcctggtacc gtttggtctg caccacatct ggaacgtacc | |
| | tttccagatg | |
| | cagattggtg aatacaccaa cgcagcaggt caggttttcc acggcgacat | |
| | tccgcgttat | |
| | atggcgggtg acccgactgc gggtaaactg tctggtggct tcctgttcaa | |
| | aatgtacggt | |
| | ctgccagctg ccgcaattgc tatctggcac tctgctaaac cagaaaaccg | |
| | cgcgaaagtg | |
| | ggcggtatta tgatctccgc ggcgctgacc tcgttcctga ccggtatcac | |
| | cgagccgatc | |
| | gagttctcct tcatgttcgt tgcgccgatc ctgtacatca tccacgcgat | |
| | tctggcaggc | |
| | ctggcattcc caatctgtat tcttctgggg atgcgtgacg gtacgtcgtt | |
| | ctcgcacggt | |
| | ctgatcgact tcatcgttct gtctggtaac agcagcaaac tgtggctgtt | |
| | cccgatcgtc | |
| | ggtatcggtt atgcgattgt ttactacacc atcttccgcg tgctgattaa | |
| | agcactggat | |
| | ctgaaaacgc cgggtcgtga agacgcgact gaagatgcaa aagcgacagg | |
| | taccagcgaa | |
| | atggcaccgg ctctggttgc tgcatttggt ggtaaagaaa acattactaa | |
| | cctcgacgca | |
| | tgtattaccc gtctgcgcgt cagcgttgct gatgtgtcta aagtggatca | |
| | ggccggcctg | |
| | aagaaactgg gcgcagcggg cgtagtggtt gctggttctg gtgttcaggc | |
| | gattttcggt | |
| | actaaatccg ataacctgaa aaccgagatg gatgagtaca tccgtaacca | |
| | ctaa | |
| Gene | atgtttaaga atgcatttgc taacctgcaa aagttyggta aatcgctgat | 9 |
| sequence | gctgccggta tccgtactgc ctatcgcagg tattctgctg ggcgtcggtt | |
| of | ccgcgaattt cagctggctg | |
| ptsGV12F | cccgccgttg tatcgcatgt tatggcagaa gcaggcggtt ccgtctttgc | |
| | aaacatgcca | |
| | ctgatttttg cgatcggtgt cgccctcggc tttaccaata acgatggcgt | |
| | atccgcgctg | |
| | gccgcagttg ttgcctatgg catcatggtt aaaaccatgg ccgtggttgc | |
| | gccactggta | |
| | ctgcatttac ctgctgaaga aatcgcctct aaacacctgg cggatactgg | |
| | cgtactcgga | |
| | gggattatct ccggtgcgat cgcagcgtac atgtttaacc gtttctaccg | |
| | tattaagctg | |
| | cctgagtatc ttggcttctt tgccggtaaa cgctttgtgc cgatcatttc | |
| | tggcctggct | |
| | gccatcttta ctggcgttgt gctgtccttc atttggccgc cgattggttc | |
| | tgcaatccag | |
| | accttctctc agtgggctgc ttaccagaac ccggtagttg cgtttggcat | |
| | ttacggtttc | |
| | atcgaacgtt gcctggtacc gtttggtctg caccacatct ggaacgtacc | |
| | tttccagatg | |
| | cagattggtg aatacaccaa cgcagcaggt caggttttcc acggcgacat | |
| | tccgcgttat | |
| | atggcgggtg acccgactgc gggtaaactg tctggtggct tcctgttcaa | |
| | aatgtacggt | |
| | ctgccagctg ccgcaattgc tatctggcac tctgctaaac cagaaaaccg | |
| | cgcgaaagtg | |
| | ggcggtatta tgatctccgc ggcgctgacc tcgttcctga ccggtatcac | |
| | cgagccgatc | |
| | gagttctcct tcatgttcgt tgcgccgatc ctgtacatca tccacgcgat | |
| | tctggcaggc | |
| | ctggcattcc caatctgtat tcttctgggg atgcgtgacg gtacgtcgtt | |
| | ctcgcacggt | |
| | ctgatcgact tcatcgttct gtctggtaac agcagcaaac tgtggctgtt | |
| | cccgatcgtc | |
| | ggtatcggtt atgcgattgt ttactacacc atcttccgcg tgctgattaa | |
| | agcactggat | |
| | ctgaaaacgc cgggtcgtga agacgcgact gaagatgcaa aagcgacagg | |
| | taccagcgaa | |
| | atggcaccgg ctctggttgc tgcatttggt ggtaaagaaa acattactaa | |
| | cctcgacgca | |
| | tgtattaccc gtctgcgcgt cagcgttgct gatgtgtcta aagtggatca | |
| | ggccggcctg | |
| | aagaaactgg gcgcagcggg cgtagtggtt gctggttctg gtgttcaggc | |
| | gattttcggt | |
| | actaaatccg ataacctgaa aaccgagatg gatgagtaca tccgtaacca | |
| | ctaa | |

The amino acid sequence of SEQ ID NO: 7 refers to the amino acid sequence of wild-type putative aga operon transcriptional repressor (AgaR). In one embodiment of the present invention, the gene of SEQ ID NO: 3 encoding the wild-type putative aga operon transcriptional repressor (AgaR) is mutated to be inactivated so that tagatose aldolase (KbaY) may be expressed.

The putative aga operon transcriptional repressor (AgaR) is predicted to have the function of inhibiting the aga operon for N-acetyl galactosamine transport and metabolism.

In one embodiment of the present invention, the gene of SEQ ID NO: 3 encoding the putative aga operon transcriptional repressor (AgaR) may comprise SEQ ID NO: 10.

TABLE 6

| Sequence | | SEQ ID |
|---|---|---|
| name | Sequence | NO. |
| Amino acid | MSNTDASGEK RVTGTSERRE QIIQRLRQQG SVQVNDLSAL | 7 |
| sequence | YGVSTVTIRN DLAFLEKQGI AVRAYGGALI CDSTTPSVEP | |
| of AgaR WT | SVEDKSALNT AMKRSVAKAA VELIQPGHRV ILDSGTTTFE | |
| | IARLMRKHTD VIAMTNGMNV ANALLEAEGV ELLMTGGHLR | |
| | RQSQSFYGDQ AEQSLQNYHF DMLFLGVDAI DLERGVSTHN | |
| | EDEARLNRRM CEVAERIIVV TDSSKENRSS LHKIIDTQRI | |
| | DMIIVDEGIP ADSLEGLRKA GVEVILVGE | |
| Gene | atgagtaata ccgacgcttc aggtgagaag cgagtgacag | 10 |
| sequence | gcaccagcga gcgacgagaa cagatcattc agcgtctgcg | |
| of AgaR WT | acagcaaggg agtgtgcagg ttaacgatct gtcggcattg | |
| | tatggcgtat ctaccgtgac gatccgcaac gatctggcgt | |
| | ttctggaaaa gcaggggatc gctgtgcgtg cctatggtgg | |
| | cgcgttgatc tgcgatagca cgacgccgtc agtcgagcca | |
| | tcagtggaag ataaaagcgc actgaacacc gcgatgaaac | |
| | gcagcgttgc gaaagctgcc gttgagttga ttcagccagg | |
| | tcatcgggtg atcctcgatt ccgggaccac cacttttgag | |
| | attgctcgtc tgatgcgcaa gcacactgac gtaattgcga | |
| | tgaccaacgg tatgaacgtg gctaatgctt tgctggaagc | |
| | ggaaggcgtt gagctgctga tgaccggcgg gcatttgcgc | |
| | cgtcagtcgc aatcttttta cggcgatcag gctgaacaat | |
| | cgctgcaaaa ttaccacttc gatatgctgt ttcttggtgt | |
| | agatgcgatc gatctggagc gcggcgtcag cacgcataat | |
| | gaagatgaag cccgtttaaa ccgccggatg tgcgaagttg | |
| | cggaacggat catcgtagtc accgattcca gtaagttcaa | |
| | ccgctccagt ttacataaga tcattgatac tcaacgtatc | |
| | gacatgatca ttgttgatga aggcattcct gcggatagtc | |
| | tggaaggact gcgaaaggct ggggttgaag tgattctggt | |
| | cggggagtga | |

In the present invention, the amino acid or gene sequence refers to the amino acid or gene sequence of each of SEQ ID NOs: 5 to 10, and also includes an amino acid or gene sequence having a homology or identity of at least 80%, 85%, 90%, 91%, 92%, 93%, 94, 95%, 96%, 97%, 98%, or 99% to the amino acid or gene sequence of each of SEQ ID NOs: 5 to 10, and those having the function of each amino acid or gene sequence described in the present invention are included in the scope of the present invention.

As used herein, the term “inactivated” refers to the case where genes encoding proteins such as enzymes, transcription factors, or transport proteins are not expressed at all compared to those in a natural strain, a wild-type strain, or a strain before modification, or the case where these genes have no activity even if expressed. In the present invention, the inactivation is performed by gene deletion, gene truncation by insertion of a heterogeneous sequence, nonsense mutation, frameshift mutation, missense mutation, or the like so that the gene is not transcribed or even if the gene is transcribed, the target protein (amino acid) does not function.

As an example of the present invention, when the expression cassette comprises the gene mutated to inactivate the fructose-bisphosphate aldolase class 2 (FbaA) of SEQ ID NO: 5 and/or the gene mutated to inactivate the putative aga operon transcriptional repressor (AgaR) of SEQ ID NO: 7, it may comprise the genes mutated such that the two enzymes cannot be expressed or cannot function, or the two genes may be deleted so that the two enzymes cannot be expressed.

In one embodiment of the present invention, the inactivation refers to gene deletion.

Another aspect of the present invention provides a recombinant vector containing the expression cassette.

As used herein, the term “recombinant vector” refers to a recombinant DNA molecule containing a desired coding sequence and appropriate nucleic acid sequences necessary for expression of the operably linked coding sequence in a particular host cell. The promoters, enhancers, termination signals and polyadenylation signals available in eukaryotic cells are known.

As used herein, the term “operably linked” refers to a functional linkage between a gene expression regulatory sequence and another nucleotide sequence. The gene expression regulatory sequence may be one or more selected from the group consisting of an origin of replication, a promoter, and a transcription termination sequence (terminator). The transcription termination sequence may be a polyadenylation sequence (pA), and the origin of replication may be an f1 origin of replication, an SV40 origin of replication, a pMB1 origin of replication, an adeno origin of replication, an AAV origin of replication, or a BBV origin of replication, without being limited thereto.

The recombinant vector according to one embodiment of the present invention may be selected from the group consisting of plasmid vectors, cosmid vectors, and viral vectors such as bacteriophage vectors, adenovirus vectors, retrovirus vectors, and adeno-associated virus vectors. Vectors that may be used as recombinant expression vectors may be constructed based on plasmids (e.g., pcDNA series, pSC101, pGV1106, pACYC177, ColE1, pKT230, pME290, pBR322, pUC8/9, pUC6, pBD9, pHC79, pIJ61, pLAFR1, pHV14, pGEX series, pET series, pUC19, etc.), phages (e.g., λgt4λB, λ-Charon, λΔz1, M13, etc.), or viral vectors (e.g., adeno-associated virus (AAV) vector, etc.), etc., which are used in the art, without being limited thereto.

The recombinant vector of the present invention may further contain one or more selectable markers. The markers are nucleic acid sequences that have the properties that can generally be selected by chemical methods, and include all genes that can distinguish transfected cells from non-transfected cells. Examples of the markers include, but are not limited to, genes resistant to herbicides such as glyphosate, glufosinate ammonium, or phosphinothricin, and genes resistant to antibiotics such as ampicillin, kanamycin, G418, bleomycin, hygromycin, or chloramphenicol.

The recombinant vector of the present invention may be constructed using genetic recombination technology well known in the art, and site-specific DNA cleavage and ligation may be performed using enzymes generally known in the art.

Another aspect of the present invention provides a mutant strain transformed with the above-described recombinant vector.

For transformation with the recombinant vector, a transfer method widely known in the art may be used. For example, when the host cell is a prokaryotic cell, the transfer may be performed using a CaCl₂ method or an electroporation method, and when the host cell is a eukaryotic cell, the transfer may be performed using a microinjection, calcium phosphate precipitation, electroporation, liposome-mediated transfection, heat shock, or gene bombardment method, without being limited thereto.

Another aspect of the present invention provides a mutant strain comprising any one or more of 1) a gene mutated to inactivate the fructose-bisphosphate aldolase class 2 (FbaA) of SEQ ID NO: 5, 2) a gene encoding a novel phosphotransferase system G in which valine (V) at position 12 in the amino acid sequence of SEQ ID NO: 6 is substituted with phenylalanine (F), and 3) a gene mutated to inactivate the putative aga operon transcriptional repressor (AgaR) of SEQ ID NO: 7.

The mutant strain may comprise, in addition to the mutant strain transformed with the above-described recombinant vector, any one or more gene mutation sequences selected from among 1) a gene mutated to inactivate the fructose-bisphosphate aldolase class 2 (FbaA) of SEQ ID NO: 5, 2) a gene encoding a novel phosphotransferase system G in which valine (V) at position 12 in the amino acid sequence of SEQ ID NO: 6 is substituted with phenylalanine (F), and 3) a gene mutated to inactivate the putative aga operon transcriptional repressor (AgaR) of SEQ ID NO: 7, which are introduced by various mutagenesis methods.

The gene sequence included in the mutant strain may comprise any one or more gene sequences selected from among 1) a gene mutated to inactivate the fructose-bisphosphate aldolase class 2 (FbaA) of SEQ ID NO: 5, 2) a gene encoding a novel phosphotransferase system G in which valine (V) at position 12 in the amino acid sequence of SEQ ID NO: 6 is substituted with phenylalanine (F), and 3) a gene mutated to inactivate the putative aga operon transcriptional repressor (AgaR) of SEQ ID NO: 7; or
comprise 1) a gene mutated to inactivate the fructose-bisphosphate aldolase class 2 (FbaA) of SEQ ID NO: 5, and 2) a gene encoding a novel phosphotransferase system G in which valine (V) at position 12 in the amino acid sequence of SEQ ID NO: 6 is substituted with phenylalanine (F); or comprise 1) a gene mutated to inactivate the fructose-bisphosphate aldolase class 2 (FbaA) of SEQ ID NO: 5, and 3) a gene mutated to inactivate the putative aga operon transcriptional repressor (AgaR) of SEQ ID NO: 7; or comprise 2) a gene encoding a novel phosphotransferase system G in which valine (V) at position 12 in the amino acid sequence of SEQ ID NO: 6 is substituted with phenylalanine (F), and 3) a gene mutated to inactivate the putative aga operon transcriptional repressor (AgaR) of SEQ ID NO: 7; or
comprise 1) a gene mutated to inactivate the fructose-bisphosphate aldolase class 2 (FbaA) of SEQ ID NO: 5, 2) a gene encoding a novel phosphotransferase system G in which valine (V) at position 12 in the amino acid sequence of SEQ ID NO: 6 is substituted with phenylalanine (F), and 3) a gene mutated to inactivate the putative aga operon transcriptional repressor (AgaR) of SEQ ID NO: 7.

The host of the mutant strain may be any host known in the art. Prokaryotic cells usable as the host include, for example, E. coli JM109, E. coli BL21, E. coli RR1, E. coli LE392, E. coli B, E. coli X 1776, E. coli W3110, Bacillus sp. strains such as Bacillus subtilis and Bacillus thuringiensis, and Enterobacteriaceae strains such as Salmonella typhimurium, Serratia marcescens and various Pseudomonas species. Examples of host cells that may be used for transformation into eukaryotic cells include yeast (Saccharomyces cerevisiae), insect cells, plant cells, and animal cells such as SP2/0, CHO (Chinese hamster ovary) K1, CHO DG44, PER.C6, W138, BHK, COS-7, 293, HepG2, Huh7, 3T3, RIN and MDCK cell lines. Specifically, the host of the mutant strain may be E. coli.

In one embodiment of the present invention, the mutant strain is able to metabolize D-tagatose. Specifically, the recombinant strain is able to metabolize D-tagatose because it contains the above-described expression cassette or mutations. The phrase “able to metabolize D-tagatose” means utilizing D-tagatose as an energy source.

Another aspect of the present invention provides a method for producing a strain able to metabolize D-tagatose, the method comprising a step of culturing the mutant strain in a medium containing D-tagatose.

The step of culturing is a step of culturing the mutant strain in a medium containing D-tagatose, wherein the medium may contain known components for culturing the mutant strain.

To achieve the above-described objects, an aspect of the present invention provides a composition for selecting a variant with increased activity, the composition comprising an agent capable of detecting mutation at any one or more of positions 16, 92, 95, 105, 129, 148, 193, 236, 324, 341 and 362 in the amino acid sequence of SEQ ID NO: 11.

The amino acid sequence of SEQ ID NO: 11 is tagaturonate/fructuronate epimerase (uxaE) that catalyzes the epimerization of D-tagaturonate (D-TagA) to D-fructuronate (D-FruA).

TABLE 7

| Se- | | SEQ |
|---|---|---|
| quence | | ID |
| name | Sequence | NO. |
| uxaE | MVLKVFKDHF GRGYEVYEKS YREKDSLSFF | 11 |
| | LTKGEEGKIL VVAGEKAPEG LSFFKKQRVE | |
| | GVSFFFCERN HENLEVLRKY FPDLKPVRAG | |
| | LRASFGTGDR LGITTPAHVR ALKDSGLFPI | |
| | FAQQSVRENE RTGRTWRDVL DDATWGVFQE | |
| | GYSEGFGADA DHVKRPEDLV SAAREGFTMF | |
| | TIDPSDHVRN LSKLSEREKN EMFEEILKKE | |
| | RIDRIYLGKK YTVLGERLEF DEKNLRDAAL | |
| | VYYDAIAHVD MMYQILKDET PDFDFEVSVD | |
| | ETETPTSPLF HIFVVEELRR RGVEFTNLAL | |
| | RFIGEWEKGI DYKGDLAQFE REIKMHAEIA | |
| | RMFEGYKISL HSGSDKFSVY PAFASATGGL | |
| | FHVKTAGTSY LEAVKVISMV NPELFREIYR | |
| | CALDHFEEDR KSYHISADLS KVPEVEKVKD | |
| | EDLPGLFEDI NVRQLIHVTY GSVLKDASLK | |
| | ERLFKTLEQN EELFYETVAK HIKRHVDLLK | |
| | G | |

In another embodiment of the present invention, the agent of the present invention is capable of detecting mutation at any one or more of positions 16, 105, 148 and 236 or at any one or more of positions 92, 95, 129, 193, 324, 341 and 362 in the amino acid sequence of SEQ ID NO: 11. More specifically, the agent is capable of detecting mutations at positions 16, 105, 148 and 236 or at positions 92, 95, 129, 193, 324, 341 and 362 in the amino acid sequence of SEQ ID NO: 11.

In one embodiment of the present invention, the positions may be V16, R92, F95, T105, N129, F148, K193, R236, K324, H341 and H362. More specifically, the mutations may be V16A, R92S, F95I, T105A, N129Y, F148S, K193E, R236S, K324N, H341L and H362I.

Detection of the mutations at the above-described positions may be achieved by analyzing the protein sequence or the nucleotide sequence encoding the mutations. The analysis method may be any conventional expression level analysis method used in the art, and examples of the analysis method include, but are not limited to, RT-PCR, competitive RT-PCR, real-time RT-PCR, RNase protection assay (RPA), Northern blotting, DNA microarray chip assay, and the like.

According to one embodiment, the agent may be used in any one of polymerase chain reaction, reverse transcription-polymerase chain reaction (RT-PCR), competitive reverse transcription-polymerase chain reaction (competitive RT-PCR), RNase protection assay (RNase, S1 nuclease assay), in situ hybridization, nucleic acid microarray assay, next-generation sequencing, and Northern blotting.

Primers may be used in the polymerase chain reaction. The “primers” are short single-stranded oligonucleotides that serve as a starting point for DNA synthesis. The primers bind specifically to the template polynucleotide under appropriate buffer and temperature conditions, and DNA is synthesized as DNA polymerase adds and links a nucleoside triphosphate having a base complementary to the template DNA to each primer. Primers generally each consist of a nucleotide sequence of 15 to 30 nucleotides, and the melting temperature (Tm) at which they bind to the template strand varies depending on the composition and length of the nucleotide sequence.

The primer sequences do not need to be completely complementary to a portion of the nucleotide sequence of the template, and it is sufficient for the primer sequences to have sufficient complementarity to the template to the extent that they can hybridize with the template and perform their own function. Therefore, the primer sequences do not need to be perfectly complementary to the variant gene sequence for measuring the expression level of the gene encoding the variant of the present invention, and it is sufficient that the primer sequences have a length and complementarity suitable for the purpose of measuring the level of mRNA of the variant by amplifying a specific region of the mRNA or cDNA of the variant through DNA synthesis. The primers for the amplification reaction are a set (pair) of primers that bind complementarily to a template (or sense) and an opposite side (antisense), respectively, at both ends of a specific region of the mRNA of the variant to be amplified. Those skilled in the art can easily design the primers with reference to the mRNA or cDNA nucleotide sequence of the variant.

The microarray assay may use as a probe any one selected from the group consisting of variant gene mRNA, variants, and fragments thereof.

As used herein, the term “probe” refers to a fragment of a polynucleotide, such as RNA or DNA, capable of specifically binding to mRNA or complementary DNA (cDNA) of a specific gene and having a length from several to several hundreds of base pairs. Since the probe is labeled, the probe can be used to check the presence or absence of the target mRNA or cDNA to be bound or the expression level thereof. For the purpose of the present invention, a probe complementary to the mRNA of the variant may be used to diagnose an infectious inflammatory disease by measuring the mRNA expression level of the variant through hybridization with a sample from a subject. The choice of probes and hybridization conditions may be appropriately made according to techniques known in the art.

Another aspect of the present invention provides a kit for selecting a variant with increased activity, the kit comprising the composition for selecting a variant.

Another aspect of the present invention provides a method of providing information for selecting a variant with increased activity, the method comprising steps of: detecting mutation from a sample using the composition; and checking, based on the detected mutation, whether the sample is a variant with increased activity.

The method of detecting the mutation is as described above.

The method may comprise detecting mutation in a sample, and selecting the sample as a variant with increased activity when the sample includes the mutation site disclosed in the present invention.

### MODE FOR INVENTION

Hereinafter, one or more specific embodiments will be described in more detail with reference to examples. However, these examples are intended to illustrate one or more embodiments and the scope of the present invention is not limited to these examples.

### Example 1: Generation of Mutant Strains Able to Metabolize Tagatose

### Example 1-1. Generation of Mutant Strains

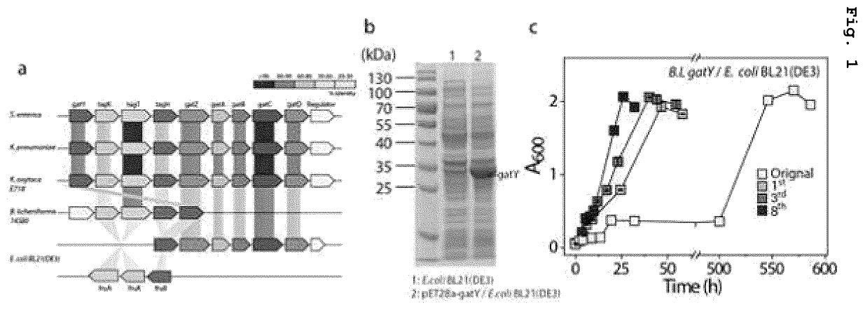

As a result of comparing the gene clusters between four strains (S. enterica, K. pneumociae, K. oxytoca, and B. licheniformis) capable of tagatose utilization and E. coli, it was confirmed that E. coli, a non-tagatose-utilizing strain, lacks the gatY gene, which is a tagatose-1,6-BP aldolase, in the fructose operon (including fruBKA) (FIG. 1a).

The gatY gene from B. licheniformis was transformed into E. coli and then subjected to adaptive evolution in tagatose medium. After about 500 hours of incubation, the strain was found to grow and subcultured continuously, and the strain whose growth rate no longer increased was subjected to whole-genome sequencing to identify mutation sites (fruK, Cra binding site, and T7RNAP promoter) (FIGS. 1b and 1c), thereby generating mutant strains.

### Experimental Example 1: Analysis of Activity of Mutant Strain

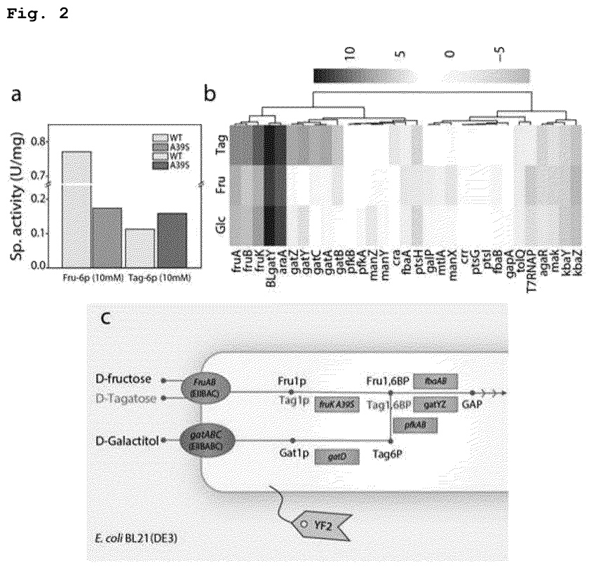

As a result of comparing the kinase activities of the wild-type strain and the mutant strain to identify factors involved in tagatose utilization, it was confirmed that the mutant strain, it was confirmed that the mutant strain showed decreased activity for Fru-6p and increased activity for Tag-6p (FIG. 2a).

After completion of adaptive evolution and whole-genome sequencing, the present inventors analyzed the mRNA expression levels of genes in media containing three sugar sources (Glc (glucose), Fru (fructose), and Tag (tagatose)) for the mutant strain (a strain in which three sites were mutated) by qRT-PCR. As a result, it was confirmed that the expression level of FruK_A39S was higher in the tagatose medium than in the other media (FIG. 2b). Thereby, it was predicted that the mutant strain would have the activity of the tagatose metabolic pathway (FIG. 2c).

### Experimental Example 2: Examination of Growth of Mutant Strain

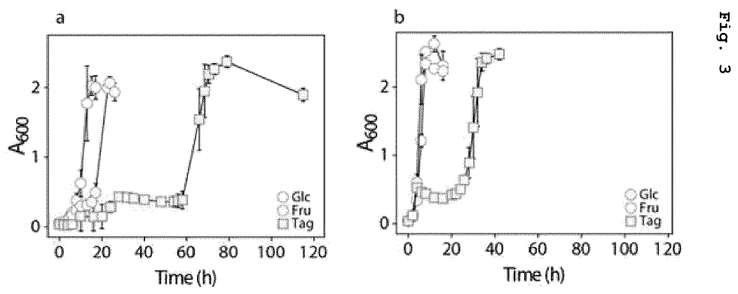

As a result of examining the growth profile of the mutant strain in which the fruK_A39S mutation was introduced and the Cra gene-binding site was deleted (a strain in which two sites were mutated), it could be confirmed that the growth of the mutant strain was better when tagatose was used as a sugar source (FIG. 3a).

In addition, as a result of examining the growth profile of the mutant strain in which the fruK_A39S mutation was introduced, the Cra gene-binding site was deleted and the T7RNAP core promoter region was mutated (a strain in which three sites were mutated), it could be confirmed that the growth of the mutant strain was better when tagatose was used as a sugar source (FIG. 3b).

### Example 2: Generation of Mutant Strain Unable to Metabolize Fructose and Able to Metabolize Tagatose

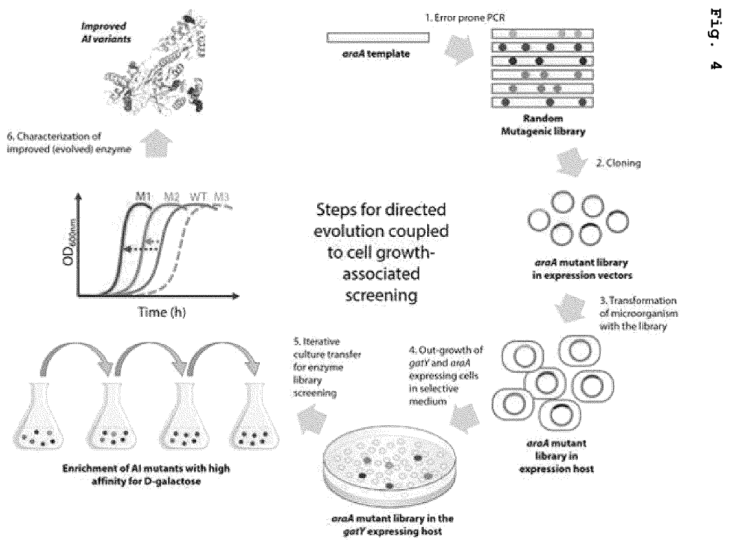

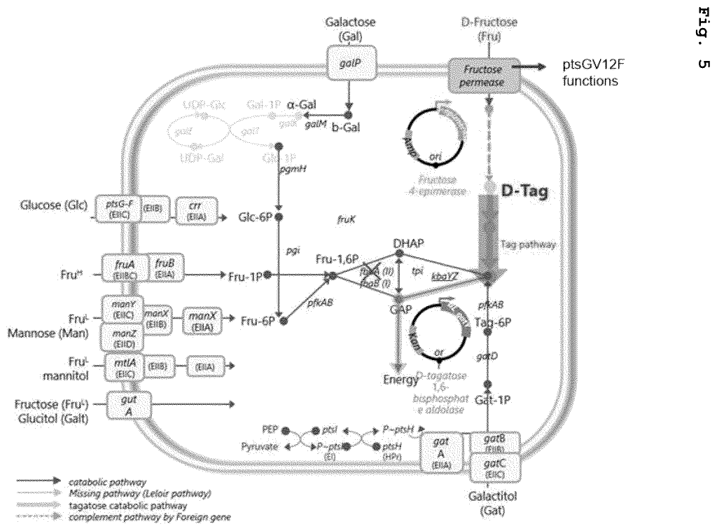

E. coli metabolizes tagatose through a phosphotransferase system (PTS) common to fructose. Thus, if the transporter (fruAB) or kinase (fruK) gene contained in the main PTS for fructose is selected to generate a strain that does not metabolize fructose, the strain can also not utilize tagatose. For this reason, the present inventors attempted to select genes that play an important role in fructose utilization while fructose metabolism does not overlap with tagatose metabolism. Therefore, the present inventors attempted to generate a strain with delayed fructose utilization by deleting aldolase FbaA, which is a key gene that allows metabolites to enter the glycolysis pathway after being phosphorylated by transporters and kinases (FIGS. 4 and 5).

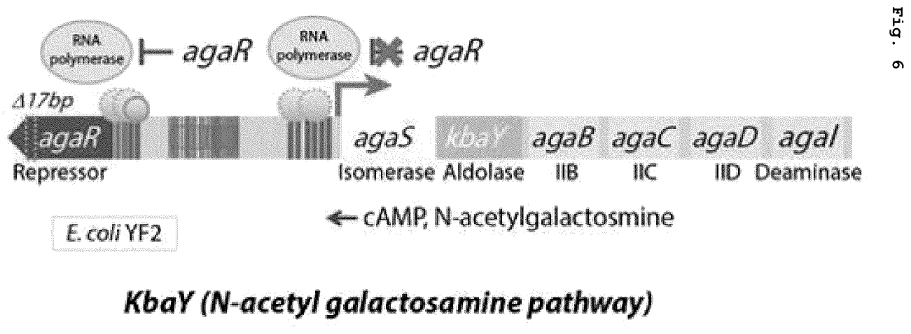

The gatY gene from B. licheniformis was transformed into E. coli which was then subjected to adaptive evolution in tagatose medium, and the resulting strain was cured. The cured strain was subjected again to adaptive evolution in tagatose medium and then to whole-genome sequencing. As a result, it was confirmed that the AgaR site was inactivated (FIG. 6).

### Experimental Example 3: Analysis of Activity of Mutant Strain

It was confirmed that the strain with FbaA gene deletion, ptsGV12F gene mutation and AgaR gene deletion had reduced ability to metabolize fructose compared to wild-type E. coli and utilized tagatose.

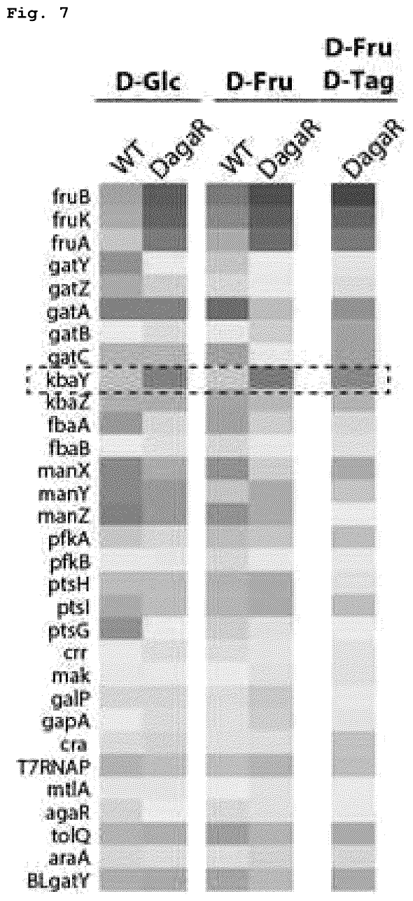

Each of the strain with AgaR gene deletion and the wild-type strain was cultured in media containing three sugar sources (Glc (glucose), Fru (fructose), and Tag (tagatose)), and the mRNA expression levels of genes were analyzed by qRT-PCR. As a result, as shown in FIG. 7, it was confirmed that, when the AgaR gene was deleted, KbaY tagatose-1,6 bisphosphate aldolase was overexpressed, thereby facilitating tagatose utilization.

### Experimental Example 4: Examination of Growth of Mutant Strain

As a result of culturing a strain with a mutated fructose metabolic gene in fructose plus tagatose media, it was confirmed that the utilization of the strain for fructose as a substrate decreased and the strain utilized tagatose as a substrate.

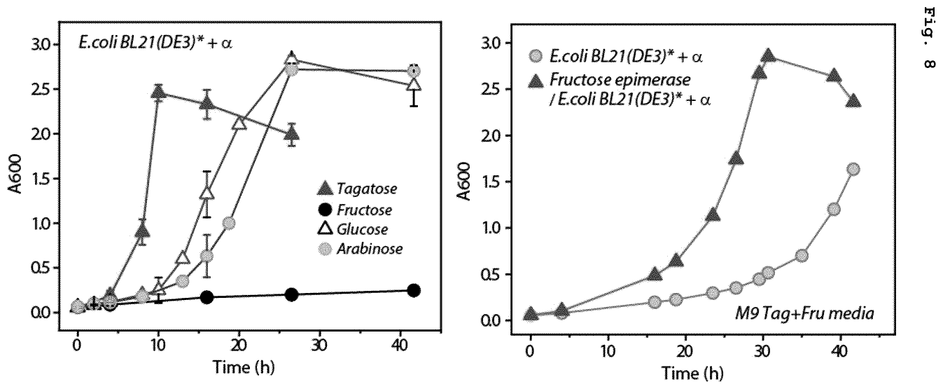

As a result, as shown in FIG. 8, it was confirmed that, when fructose epimerase was introduced into this strain and the strain was grown in fructose medium, the strain grew in an enzyme activity-dependent manner.

### Example 3: Construction of Libraries

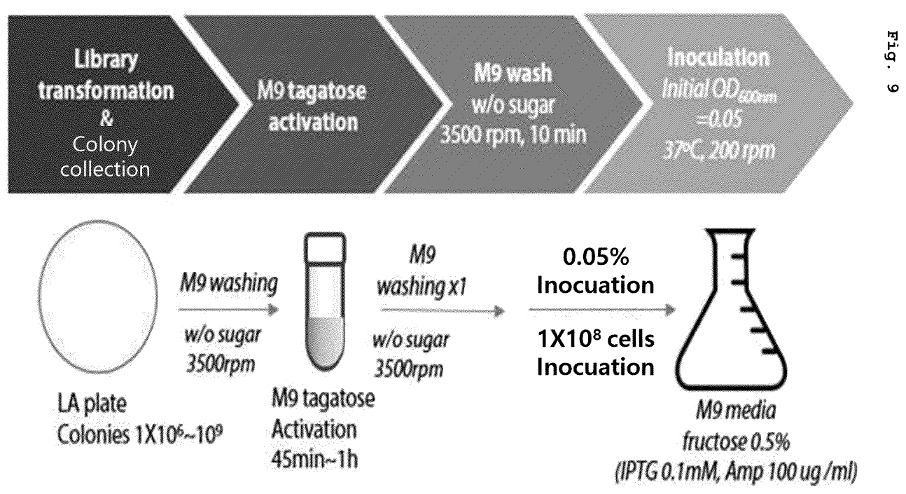

As shown in FIG. 9, in order to induce mutation in the D-fructose epimerase uxaE gene, error-prone PCR was performed using a PCR random mutagenesis kit (Clontech, USA). 50 ng of mutation-induced PCR library DNA was transformed into E. coli BL21(DE3) with a mutated fructose metabolic pathway, and the strain was cultured in minimal (M9) medium containing 0.5% fructose. Thereafter, the formed colonies were collected and plasmids were extracted therefrom using a plasmid purification kit. Among them, some plasmids were sequenced, and the genetic diversity of the library was examined (Table 8).

TABLE 8

| | Lib1 | Lib2 | Lib3 |
|---|---|---|---|
| 1 | V16A | R92S | V16A |
| 2 | T105A | F95I | T105A |
| 3 | F148S | N129Y | F148S |
| 4 | R236S | K193E | R236S |
| 5 | | K324N | |
| 6 | | H341L | |
| 7 | | H362I | |

### Example 4: Selection and Analysis of Activity of Variants

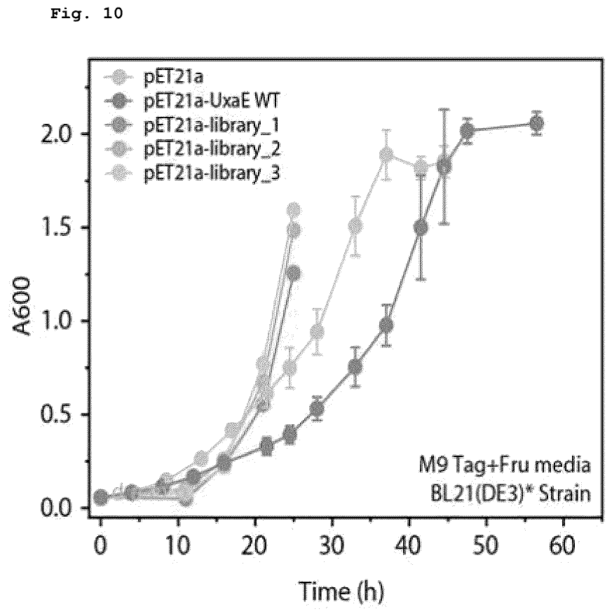

BL21 (DE3) with a modified fructose metabolic pathway transformed with the glycotransferase library gene-modified fructose epimerase gene pET-21a(+)-uxaE library DNA confirmed to be diverse was cultured in minimal (M9) medium containing 0.5% D-fructose and 0.2 mM (final concentration) IPTG, and the growth of the strain was examined. The results are shown in FIG. 10.

As shown in FIG. 10, it was confirmed that the level of growth of the strain containing the mutant library gene was different that of the wild-type strain. Thereby, it was confirmed that the ability to use D-fructose as a carbon source may vary depending on the introduction and mutation of the uxaE gene.

### Example 3: Analysis of Activity of Variant According to Structural Prediction

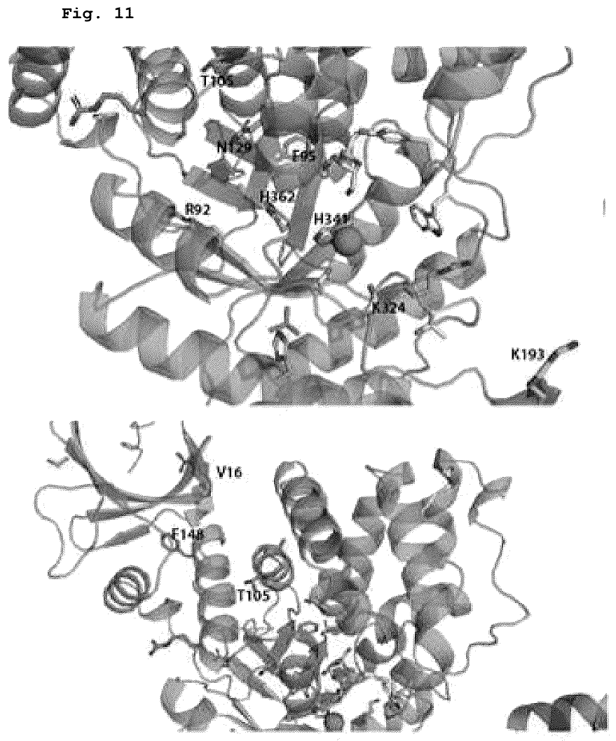

The 11 mutation sites identified above are adjacent to the metal-binding site, and thus they appear to have an effect on improving the activity of the protein by forming a tertiary structure of the protein (FIG. 11).

So far, the present invention has been described with reference to the embodiments. Those of ordinary skill in the art to which the present invention pertains will appreciate that the present disclosure may be embodied in modified forms without departing from the essential characteristics of the present invention. Therefore, the disclosed embodiments should be considered from an illustrative point of view, not from a restrictive point of view. The scope of the present invention is defined by the claims rather than the foregoing description, and all differences within the scope equivalent thereto should be construed as being included in the present invention.

1. An expression cassette comprising a gene encoding a novel fructose-1-phosphate kinase in which alanine (A) at position 39 in the amino acid sequence of SEQ ID NO: 1 is substituted with serine (S).

2. The expression cassette of claim 1, wherein the gene encoding the novel fructose-1-phosphate kinase is SEQ ID NO: 2.

3. The expression cassette of claim 1, wherein the expression cassette is one in which a Cra-binding site is deleted.

4. The expression cassette of claim 3, wherein the deletion of the Cra-binding site corresponds to deletion of the sequence of SEQ ID NO: 3 in the expression cassette.

5. The expression cassette of claim 1, further comprising a mutation sequence between a sequence encoding LacI and a sequence encoding T7 RNAP.

6. The expression cassette of claim 5, wherein the mutation sequence between the sequence encoding LacI and the sequence encoding T7 RNAP is mutation in a T7 RNAP core promoter region.

7. The expression cassette of claim 5, wherein the mutation sequence between the sequence encoding LacI and the sequence encoding T7 RNAP is SEQ ID NO: 4.

8-13. (canceled)

14. An expression cassette comprising:
a gene mutated to inactivate the fructose-bisphosphate aldolase class 2 (FbaA) of SEQ ID NO: 5;
a gene encoding a novel phosphotransferase system G in which valine (V) at position 12 in the amino acid sequence of SEQ ID NO: 6 is substituted with phenylalanine (F); and
a gene mutated to inactivate the putative aga operon transcriptional repressor (AgaR) of SEQ ID NO: 7.

15. The expression cassette of claim 14, wherein the gene encoding the novel phosphotransferase system G (PstG) is SEQ ID NO: 9.

16. The expression cassette of claim 14, wherein the inactivation of the putative aga operon transcriptional repressor causes tagatose aldolase (KbaY) to be expressed.

17. The expression cassette of claim 14, wherein the inactivation is achieved by deletion of the gene.

18-23. (canceled)

24. A composition for selecting a variant with increased activity, the composition comprising an agent capable of detecting mutation at any one or more of positions 16, 92, 95, 105, 129, 148, 193, 236, 324, 341 and 362 in the amino acid sequence of SEQ ID NO: 11.

25. The composition of claim 24, wherein the mutation may be mutation at any one or more positions selected from among V16, R92, F95, T105, N129, F148, K193, R236, K324, H341, and H362.

26. The composition of claim 24, wherein the mutation is any one or more of V16A, R92S, F95I, T105A, N129Y, F148S, K193F, R236S, K324N, H341L, and FH362I.

27. The composition of claim 24, wherein the agent is used in any one of polymerase chain reaction, reverse transcription-polymerase chain reaction (RI-PCR), competitive reverse transcription-polymerase chain reaction (competitive RT-PCR), RNase protection assay (RNase, S1 nuclease assay), in situ hybridization, nucleic acid microarray assay, next-generation sequencing, and Northern blotting.

28-29. (canceled)
